(19) United States
(12) Patent Application Publication
(10) Pub. No.: US 2009/0156609 A1
(43) Pub. Date: Jun. 18, 2009

(54) TREATMENT OF POST-TRAUMATIC STRESS DISORDER WITH TETRAHYDROINDOLONE ARYLPIPERZAINE COMPOUNDS

(76) Inventors: David Helton, Foothill Ranch, CA (US); David Fick, Newport Beach, CA (US); Ernest Pfadenhauer, Costa Mesa, CA (US)

Correspondence Address: SHELDON MAK ROSE & ANDERSON PC, 100 Corson Street, Third Floor, PASADENA, CA, 91103-3842, US

(21) Appl. No.: 12/268,152

(22) Filed: Nov. 10, 2008

Related U.S. Application Data
(60) Provisional application No. 60/986,906, filed on Nov. 9, 2007.

(51) Int. Cl. A61K 31/506; A61K 31/496

(52) U.S. Cl. 514/252.19; 514/254.09

(57) ABSTRACT

Tetrahydroindolone and aryl piperazine derivatives for use in treating post-traumatic stress disorder and acute stress disorder.

### BACKGROUND

Posttraumatic stress disorder (PTSD) is a chronic psychiatric disorder that is triggered by extreme psychological trauma, including rape, exposure to warfare, and even cancer. It was described in veterans of the American Civil War, and has been called “shell shock,” “combat neurosis,” and “operational fatigue.” Symptoms of the disorder may include nightmares, flashbacks, emotional detachment or numbing of feelings (emotional self-mortification or dissociation), insomnia, avoidance of reminders and extreme distress when exposed to the reminders (“triggers”), loss of appetite, irritability, hypervigilance, memory loss (may appear as difficulty paying attention), excessive startle response, clinical depression, and anxiety. The lifetime prevalence of PTSD in the U.S. is approximately 8% of the U.S. population. The rate among former combat soldiers runs much higher.

Current treatment options for PTSD include patient education, social support, and anxiety management through psychotherapy and psychophammacologic intervention. Patient education and social support are important initial interventions to engage the patient and mitigate the impact of the traumatic event. However, the mainstay of treatment is psychopharmacologic and psychotherapeutic intervention.

Medications for treating PTSD include antidepressants and antipsychotic drugs. Paroxetine (Paxil) and sertraline (Zoloft) are currently the only medications that have been approved by the U.S. Food and Drug Administration for the treatment of PTSD. Of the patients who received 20 mg or 40 mg of paroxetine, 62 percent and 54 percent, respectively, responded positively compared with 37 percent of patients who received a placebo.

### SUMMARY

While present treatments help some PTSD sufferers, the effectiveness of present medications in treating symptoms of the disorder vary from patient to patient. The side effects of these drugs may also be troublesome enough for some PTSD sufferers to discontinue their treatment. There is therefore a need for additional and improved therapeutic agents for treating PTSD.

The present invention provides methods of treating post-traumatic stress disorder or acute stress disorder in subjects, in particular human subjects, by administering to such subjects a therapeutic dose of a pharmaceutical composition comprising a compound having the following formula:

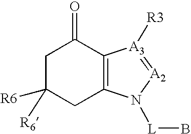

where:
(a) A₂ and A₃ are C or N;
(b) R₃ is hydrogen, alkyl, aralky, heteroaralkyl, alkenyl, aralkenyl, heteroaralkenyl, aryl, heteroaryl, or does not exist when A₃ is N;
(c) R₆ is hydrogen, alkyl, aralkyl, heteroaralkyl, aryl or heteroaryl;
(d) R₆ is hydrogen unless R₆ is alkyl, in which case R₆ is hydrogen or the same alkyl as R₆;
(e) L is a linker group; and
(f) B has the following formula:

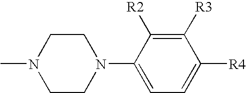

where:
(i) R2 is hydrogen, alkyl, hydroxy, halo, alkoxy, cyano, methylthio;
(ii) R3 is hydrogen, alkyl, hydroxy, methoxy, halo, alkoxy, trifluoromethyl, nitro, amino, aminocarbonyl, aminosulfonyl;
(iii) R2 and R3 can be taken together to form a 5 or 6 member aromatic or non-aromatic ring, which can contain from 0 to 3 heteroatoms selected from the group of N, O, or S;
(iv) R₄ is hydrogen, alkyl, halo, alkoxy, perfluoroalkyl, perfluoroalkoxy, or nitro; and
(v) R₃ and R₄ when taken together can form a 5 or 6 membered ring and can contain one or more heteroatoms;
   and pharmaceutically acceptable salts and esters thereof.

In preferred embodiments, R₆ and R_{6′} are both hydrogen; R₂ can be hydrogen, halo, or alkoxy; R₃ can be hydrogen, alkyl, halo, alkoxy, or perfluoroalkyl; and R₄ can be alkyl, halo, alkoxy, or perfluoroalkyl. Preferably, R₃ is trifluoromethyl or a halo group. Further, in some embodiments R₃ an R₄ are halo substituents or R₂ an R₄ are halo substituents. R₂ and R₃, when taken together, can also form a naphthalene ring. The linker group is also preferably a straight chain alkyl group of the formula —(CH₂)ₘ—, where m is an integer between 1 and 6. Preferred compositions include one or more pharmaceutically acceptable excipients.

Preferred compounds for use in the present methods include the following:
1-{2-[4-(3-Chlorophenyl)piperazin-1-yl]ethyl}-1,5,6,7-tetrahydroindol-4-one;
1-{4-[4-(4-Fluorophenyl)piperazin-1-yl]butyl}-1,5,6,7-tetrahydroindol-4-one;
1-{4-[4-(4-Bromophenyl)piperazin-1-yl]butyl}-1,5,6,7-tetrahydroindol-4-one;
1-{4-[4-(3-Trifluoromethylphenyl)piperazin-1-yl]butyl}-1,5,6,7-tetrahydroindol-4-one;
1-{2-[4-(3-Trifluoromethylphenyl)piperazin-1-yl]ethyl}-1,5,6,7-tetrahydroindol-4-one;
1-{3-[4-(3-Chlorophenyl)piperazin-1-yl]propyl}-1,5,6,7-tetrahydroindol-4-one;
1-{3-[4-(3-Trifluoromethylphenyl)piperazin-1-yl]propyl}-1,5,6,7-tetrahydroindol-4-one;
1-{4-[4-(3,4-dichlorophenyl)piperazin-1-yl]butyl}-1,5,6,7-tetrahydroindol-4-one;
1-{4-[4-(3-Chloro-4-fluorophenyl)piperazin-1-yl]butyl}-1,5,6,7-tetrahydroindol-4-one;
1-{4-[4-(2,4-Dichlorophenyl)piperazin-1-yl]butyl}-1,5,6,7-tetrahydroindol-4-one; and
1-{3-[4-(3,4-Dichlorophenyl)piperazin-1-yl]propyl}-1,5,6,7-tetrahydroindol-4-one.

A therapeutic dose of the present composition can be administered by any of a number of routes, including intravenous infusion, oral, topical, intraperitoneal, intravesical, transdermal, nasal, rectal, vaginal, intramuscular, intradermal, subcutaneous and intrathecal routes. The route of administration can influence the therapeutic dose of the present composition, as will be understood by one of skill in the art, which can be in the range of 0.0001 mg/kg to 60 mg/kg. Preferably, the present compositions are administered in a dose in the range of 0.3 mg/kg to 10 mg/kg.

### FIGURES

FIG. 1 is a bar graph showing the results of tests involving different concentrations of compound A in the PPI preclinical model.

FIG. 2 is a bar graph showing the results of tests involving Compound A in the contextual fear conditioning in an open field preclinical model.

FIG. 3 is a bar graph showing the results of tests involving Compound A in the development of contextual fear conditioning in an open field model preclinical model.

### DESCRIPTION

### Definitions

As used herein, the following terms and variations thereof have the meanings given below, unless a different meaning is clearly intended by the context in which such term is used.

“Acute stress disorder,” also referred to herein as ASD, refers to a disorder defined by criteria set forth in the Diagnostic and Statistical Manual of Mental Disorders (DSM-IV). Acute stress disorder symptoms include those set forth below for post-traumatic stress disorder, but appear within the first month following exposure to a traumatic event. If there is no improvement of symptoms after a month, PTSD is diagnosed.

“Alkyl” refers to saturated aliphatic groups including straight-chain, branched-chain, and cyclic groups, all of which can be optionally substituted. Preferred alkyl groups contain 1 to 10 carbon atoms. Suitable alkyl groups include methyl, ethyl, and the like, and can be optionally substituted. The term “heteroalkyl” refers to carbon-containing straight-chained, branch-chained and cyclic groups, all of which can be optionally substituted, containing at least one O, N or S heteroatom. The term “alkoxy” refers to the ether —O-alkyl, where alkyl is defined as above.

“Alkenyl” refers to unsaturated groups which contain at least one carbon-carbon double bond and includes straight-chain, branched-chain, and cyclic groups, all of which can be optionally substituted. Preferable alkenyl groups have 2 to 10 carbon atoms. The term “heteroalkenyl” refers to unsaturated groups which contain at least one carbon-carbon double bond and includes straight-chained, branch-chained and cyclic groups, all of which can be optionally substituted, containing at least one O, N or S heteroatom.

“Aryl” refers to aromatic groups that have at least one ring having a conjugated, pi-electron system and includes carboxcyclic aryl and biaryl, both of which can be optionally substituted. Preferred aryl groups have 6 to 10 carbon atoms. The term “aralkyl” refers to an alkyl group substituted with an aryl group. Suitable aralkyl groups include benzyl and the like; these groups can be optionally substituted. The term “aralkenyl” refers to an alkenyl group substituted with an aryl group. The term “heteroaryl” refers to carbon-containing 5-14 membered cyclic unsaturated radicals containing one, two, three, or four O, N, or S heteroatoms and having 6, 10, or 14 π-electrons delocalized in one or more rings, e.g., pyridine, oxazole, indole, thiazole, isoxazole, pyrazole, pyrrole, each of which can be optionally substituted as discussed above.

“Derivative” refers to a compound that is modified or partially substituted with another component.

“Hydrocarbyl” refers to a hydrocarbon chain, which can be optionally substituted or provided with other substitutions known to the art.

“Optionally substituted” refers to one or more substituents which can be, without limitation, alkyl, aryl, amino, hydroxy, alkoxy, aryloxy, alkylamino, arylamino, alkylthio, arylthio, or oxo, cyano, acetoxy, or halo moieties.

“Patient,” “subject,” and the like with reference to individuals that can be treated with the present compounds and/or pharmaceutical compositions refer to humans and other mammals.

“Post-traumatic stress disorder,” also referred as PTSD, is a disorder which can be diagnosed by a trained professional in view of the diagnostic criteria set forth in Table 2 below. PTSD is divided into three categories: (1) Acute PTSD, which subsides within three months; (2) Chronic PTSD, which is diagnosed if symptoms persist longer than three months; and (3) Delayed-onset PTSD, which may occur months, years or even decades after the traumatic event.

“Sulfonyl” refers to the group —S(O₂)—. The term “halo” refers to fluoro-, chloro-, bromo-, or iodo-substitutions. The term “alkanoyl” refers to the group —C(O)R, where R is alkyl. The term “aroyl” refers to the group —C(O)R, where R is aryl. Similar compound radicals involving a carbonyl group and other groups are defined by analogy. The term “aminocarbonyl” refers to the group —NHC(O)—. The term “oxycarbonyl” refers to the group —OC(O)—. The term “heteroaralkyl” refers to an alkyl group substituted with a heteroaryl group. Similarly, the term “heteroaralkenyl” refers to an alkenyl group substituted with a heteroaryl group.

As used herein, the term “comprise” and variations of the term, such as “comprising” and “comprises,” are not intended to exclude other additives, components, integers or steps. The terms “a,” “an,” and “the” and similar referents used herein are to be construed to cover both the singular and the plural unless their usage in context indicates otherwise.

### Compounds

The compounds of the present invention have the general schematic structure {A}-L-{B}, where the A moiety is a bicyclic ring structure such as tetrahydroindolone or a tetrahydroindolone derivative, L is a hydrocarbyl chain linker, and the B moiety is an arylpiperazine or arylpiperazine derivative, as described below.

### Tetrahydroindolone Moiety

In one embodiment of the present invention, A is an 8-10 atom bicyclic moiety in which the five-aromatic membered ring has 1 to 2 nitrogen atoms, the bicyclic moiety having the structure of formula (I):

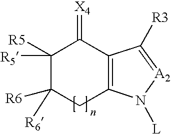

where:
(a) formula I is bonded to a hydrocarbyl linker L;
(b) A₂ is C or N;
(c) R₃ is hydrogen, alkyl, aralky, heteroaralkyl, heteroalkyl, alkenyl, aralkenyl, heteroaralkenyl, heteroalkenyl, aryl, or heteroaryl;
(d) X₄ is O, S or N—OH;
(e) R₅ is hydrogen, alkyl, aralkyl, heteroaralkyl, alkanoyl, aroyl, heteroaroyl, aralkanoyl, heteroaralkanoyl, NH₂, NH Q₁, NQ₁Q₂, OH, OQ₁, or SQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain 1 other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(f) R₅ is hydrogen unless R₅ is alkyl, in which case R₅ is hydrogen or the same alkyl as R₅;
(g) R₅ and R_{5′} can be taken together as a double bond to C₅ and can be O, S, NQ₃, or C which can be substituted with one or two groups R₅, where Q₃ is alkyl, aralkyl, heteroaralkyl, aryl, heteroaryl, hydroxy, alkoxy, aryloxy, or heteroaryloxy in which the alkyl portions can be cyclic and can contain from 1 to 3 heteroatoms which can be N, O, or S;
(h) R₆ is hydrogen, alkyl, aryl, heteroaryl;
(i) R_{6′} is hydrogen unless R₆ is alkyl, in which case R_{6′} is hydrogen or the same alkyl as R₆;

(j) n is 0 to 2.

As shown in Formula (I), the moiety A has a five, six, or seven-membered saturated ring fused to a five-membered aromatic ring. The five-membered aromatic ring can have one or two nitrogen atoms as indicated, but the five-membered aromatic ring always has a nitrogen atom at the 1-position. Typically, the five-membered aromatic ring has one nitrogen atom as in tetrahydroindolone. This nitrogen atom at the 1-position is covalently bonded to the linker L. Typically A is a tetrahydroindolone moiety in which A₂ is carbon and n is 1. The tetrahydroindolone moiety can be variously substituted.

Preferably, A is a tetrahydroindolone moiety. One example of a tetrahydroindolone moiety for the moiety A is a tetrahydroindolone moiety of Formula (II), below, in which:

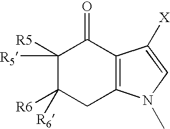

(1) X is H or CH₂N(CH₃)₂;

(2) R₅ is hydrogen, alkyl, aralkyl, heteroaralkyl, alkanoyl, aroyl, heteroaroyl, aralkanoyl, heteroaralkanoyl, NH₂, NHW₁, NQ₁Q₂, OH, OQ₁, or SQ₁, where Q₁ and Q₂ are alkyl, aralkyl, heteroaralkyl, aryl, heteroaryl, alkanoyl, aroyl, aralkanoyl, heteroaralkanoyl, or heteroaroyl in which the alkyl portions can be cyclic and can contain from 1 to 3 heteroatoms which can be N, O, or S, and where W₁ is alkyl, aralkyl, heteroaralkyl, aryl, heteroaryl, alkanoyl, aroyl, aralkanoyl, heteroaralkanoyl, or heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S;

(3) R₅ is hydrogen;

(4) R₆ is hydrogen, alkyl, aryl, heteroaryl;

(5) R₆′ is hydrogen.

Another example of a tetrahydroindolone moiety for the moiety A is the following tetrahydroindolone moiety:

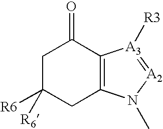

where:
(a) A₂ and A₃ are C or N;
(b) R₃ is hydrogen, alkyl, aralky, heteroaralkyl, alkenyl, aralkenyl, heteroaralkenyl, aryl, heteroaryl, or does not exist when A₃ is N;
(c) R₆ is hydrogen, alkyl, aralkyl, heteroaralkyl, aryl or heteroaryl; and
(d) R_{6′} is hydrogen unless R₆ is alkyl, in which case R_{6′} is hydrogen or the same alkyl as R₆.

In one embodiment, R₅, R_{5′}, R₆, and R_{6′}, are all hydrogen. In this embodiment, the moiety A is thus an unsubstituted tetrahydroindolone moiety. A preferred tetrahydroindolone moiety has the following formula:

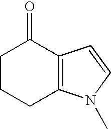

### Arylpiperazine Moiety

B is an arylpiperazine or derivative having the structure of formula (VII):

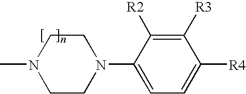

where:
(a) R2 is hydrogen, alkyl, hydroxy, halo, alkoxy, cyano, methylthio;
(b) R3 is hydrogen, alkyl, hydroxy, methoxy, halo, alkoxy, trifluoromethyl, nitro, amino, aminocarbonyl, aminosulfonyl;
(c) R2 and R3 can be taken together to form a 5 or 6 member aromatic or non-aromatic ring, which can contain from 0 to 3 heteroatoms selected from the group of N, O, or S;
(d) R₄ is hydrogen, alkyl, halo, alkoxy, perfluoroalkyl, perfluoroalkoxy, or nitro;
(e) R₃ and R₄ when taken together can form a 5 or 6 membered ring and can contain one or more heteroatoms; and
(f) n equals 1 or 2

Preferably, the aryl piperazine moiety comprises one or more of the following substitutions:
(i) R₄ is alkyl, halo, alkoxy, or perfluoroalkyl;
(ii) R₃ and R₄ when taken together are either a methylenedioxy or ethylenedioxy group.

In one embodiment, B is a m-trifluoromethylphenylpiperazinyl moiety:

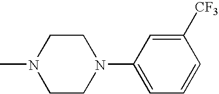

In another embodiment, B is a m-chlorophenylpiperazinyl moiety:

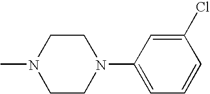

In yet another embodiment, B is an o-methoxyphenylpiperazinyl moiety:

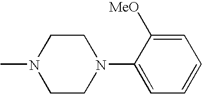

In another embodiment, B is a piperazine ring or derivative linked to a 6-member heterocyclic ring containing 1 to 3 N, having the structural formula (VIII):

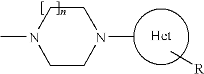

Wherein n=1 or 2 and the 6-member heterocyclic ring (Het) can be 2-pyridyl, 4-pyridyl, 2-pyrimidyl, 4-pyrimidyl, 2-pyrazinyl, or 2-triazinyl. The heterocyclic ring can also be substituted where R can be halo, alkyl, cyano, trifluoromethyl, alkoxy, amino, alkylamino, or dialkyamino.

In one embodiment, B is a 2-pyrimidylpiperazinyl moiety:

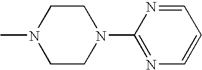

In another embodiment, B is a 1-pyrimidin-2-yl-[1,4]diazepane moiety:

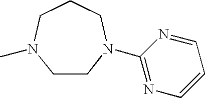

In yet another embodiment, B is a piperazine ring or derivative linked to a bicyclic moiety having the structural formula (IX):

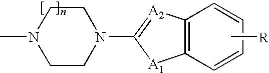

where:
(a) A₁ is N, O, or S, and when it is N, it can be further substituted with Z, which is alkyl, aralkyl, heteroaralky, or heteroalkyl.
(b) A2 is C or N;
(c) and n is 1 or 2
(d) R is hydrogen, alkyl, NH2, NHQ1, NQ1 Q2, OH, OQ1, SQ1, halo, nitro, cyano, or trifluoromethyl where Q1 and Q2 are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q1 and Q2 are present together and are alkyl, they can be taken together to form a 5- or 6-membered ring which can contain 1 other heteroatom which can be N, O, or S, of which the N can be further substituted with Y2, where Y2 is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S.

In another embodiment, B is piperazine ring or derivative linked to a bicyclic moiety having the structure (X) below:

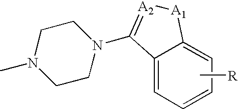

where:
(a) A1 is N, O, or S, and when it is N, it can be further substituted with Z, which in alkyl, aralkyl, heteroaralky, or heteroalkyl.
(b) A2 is C or N;
(c) and n is 1 or 2
(d) R is hydrogen, alkyl, NH2, NHQ1, NQ1 Q2, OH, OQ1, SQ1, halo, nitro, cyano, or trifluoromethyl where Q1 and Q2 are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q1 and Q2 are present together and are alkyl, they can be taken together to form a 5- or 6-membered ring which may contain 1 other heteroatom which can be N, O, or S, of which the N may be further substituted with Y2, where Y2 is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S.

In another embodiment, B is a piperazine ring or derivative linked to a bicyclic moiety having the structural formula (XI):

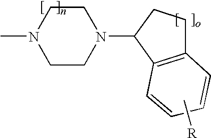

where:
(a) o is 1 to 3;
(b) n is 1 or 2; and
(c) R is hydrogen, alkyl, NH2, NHQ1, NQ1 Q2, OH, OQ1, SQ1, nitro, cyano, trifluoromethyl, or halo where Q1 and Q2 are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q1 and Q2 are present together and are alkyl, they can be taken together to form a 5- or 6-membered ring which can contain 1 other heteroatom which can be N, O, or S, of which the N can be further substituted with Y2, where Y2 is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S.

Generally, any moiety A can be combined with any linker L and any moiety B to produce a composite compound according to the present invention. However, in one embodiment the composite compounds of the present invention include, but are not limited to, the following structure:

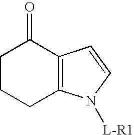

(a) wherein L is as described below; and
(b) wherein R1 is:

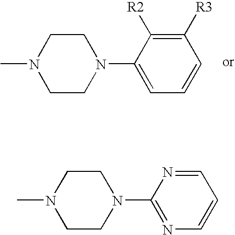

and R2 and R3 are the same or independently hydrogen, alkyl, hydroxy, methoxy, halo, alkoxy, trifluoromethyl, nitro, amino, aminocarbonyl, or aminosulfonyl.

### Linker Moiety

The linker moiety (L) used in the compounds of the present invention can be a straight chain alkyl group of the formula —(CH₂)ₘ—, where m is an integer from 1 to 6 and more preferably either 3, 4, or 5. Alternatively, the linker can be an alkyl substituted hydrocarbyl moiety of the following formula (IV):

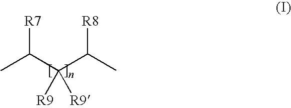

where:

(i) n is 0, 1 or 2;

(ii) R7 and R8 are hydrogen, methyl or ethyl;

(iii) R9 and R9′ are both hydrogen, methyl or ethyl;

(iv) if n is 1 and R7 or R8 is methyl or ethyl, then R9 and R9′ are hydrogen;

(v) if n is 1 and R7 and R8 are hydrogen, then R9 and R9′ are methyl or ethyl; and

(vi) if n is 2, then R9 and R9′ are hydrogen and one or both of R7 and R8 are methyl or ethyl.

The linker moiety can modulate properties of the present compounds. For example, a straight chain alkyl linker comprising two carbon atoms would provide a more rigid linkage than a longer alkyl linker. Such rigidity can produce greater specificity in target binding, while a less rigid linker moiety can produce greater potency. The solubility characteristics of the present compounds can also be affected by the nature of the linker moiety. In addition, linker groups other than those provided herein can be used to form the present compounds.

The use of a linker according to formula (IV) above is believed to provide a more rigid linkage compared to a straight chain linker moiety with the same number of carbon atoms in the chain. This allows for further control over the properties of the present compounds.

In another embodiment, linker moiety (L) can be a phenyl or a benzyl linked to a hydrocarbyl chain by group Y where group Y is located on the meta or para positions of the aromatic ring. Group Y can be nothing such that the hydrocarbyl chain is directly linked to the phenyl group. Group Y can also be an ether, thioether, carbonyl, thiocarbonyl, carboxamido, aminocarbonyl, amino, oxycarbonylamino, aminocarbonyloxy, aminocarbonylamino, oxythiocarbonylamino, aminothiocarbonyloxy, aminothiocarbonylamino, aminosulfonyl, or sulfonamido group.

The compounds of the present invention further include, but are not limited to, the following compounds:

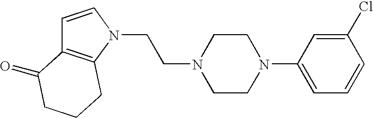

### 1-{2-[4-(3-Chlorophenyl)piperazin-1-yl]ethyl}-1,5,6,7-tetrahydroindol-4-one (Compound A);

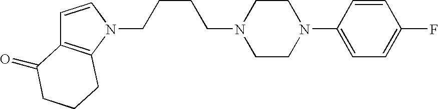

### 1-{4-[4-(4-Fluorophenyl)piperazin-1-yl]butyl}-1,5,6,7-tetrahydroindol-4-one (Compound B);

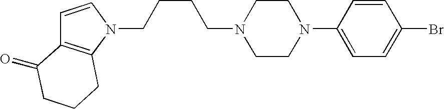

### 1-{4-[4-(4-Bromophenyl)piperazin-1-yl]butyl}-1,5,6,7-tetrahydroindol-4-one (Compound C);

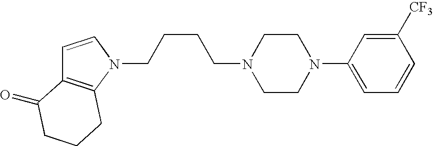

### 1-{4-[4-(3-Trifluoromethylphenyl)piperazin-1-yl]butyl}-1,5,6,7-tetrahydroindol-4-one (Compound D);

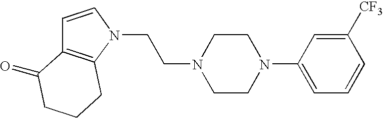

### 1-{2-[4-(3-Trifluoromethylphenyl)piperazin-1-yl]ethyl}-1,5,6,7-tetrahydroindol-4-one Compound E);

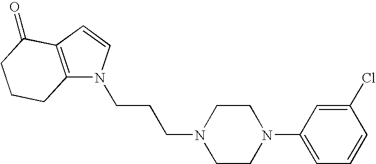

### 1-{3-[4-(3-Chlorophenyl)piperazin-1-yl]propyl}-1,5,6,7-tetrahydroindol-4-one Compound F);

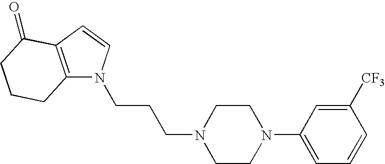

### 1-{3-[4-(3-Trifluoromethylphenyl)piperazin-1-yl]propyl}-1,5,6,7-tetrahydroindol-4-one (Compound G);

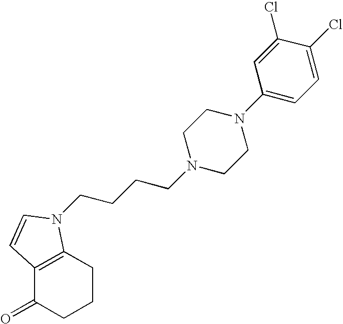

### 1-{4-[4-(3,4-dichlorophenyl)piperazin-1-yl]butyl}-1,5,6,7-tetrahydroindol-4-one (Compound H);

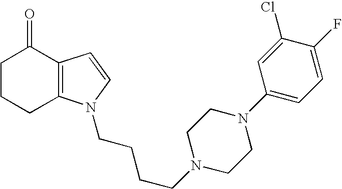

### 1-{4-[4-(3-Chloro-4-fluorophenyl)piperazin-1-yl]butyl}-1,5,6,7-tetrahydroindol-4-one (Compound I);

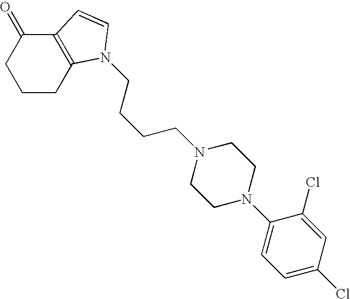

### 1-{4-[4-(2,4-Dichlorophenyl)piperazin-1-yl]butyl}-1,5,6,7-tetrahydroindol-4-one (Compound J); and

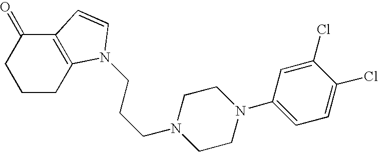

### 1-{3-[4-(3,4-Dichlorophenyl)piperazin-1-yl]propyl}-1,5,6,7-tetrahydroindol-4-one (Compound K)

Table 1 below lists further specific embodiments of the present compounds.

TABLE 1

| | |
|---|---|
| 1 | 1-{2-[4-(4-Fluorophenyl)piperazin-1-yl]ethyl}-1,5,6,7-tetrahydroindol-4-one |
| 2 | 1-{3-[4-(4-Fluorophenyl)piperazin-1-yl]propyl}-1,5,6,7-tetrahydroindol-4-one |
| 3 | 1-{5-[4-(4-Fluorophenyl)piperazin-1-yl]pentyl}-1,5,6,7-tetrahydroindol-4-one |
| 4 | 1-{2-[4-(4-Chlorophenyl)piperazin-1-yl]ethyl}-1,5,6,7-tetrahydroindol-4-one |
| 5 | 1-{3-[4-(4-Chlorophenyl)piperazin-1-yl]propyl}-1,5,6,7-tetrahydroindol-4-one |
| 6 | 1-{4-[4-(4-Chlorophenyl)piperazin-1-yl]butyl}-1,5,6,7-tetrahydroindol-4-one |
| 7 | 1-{2-[4-(4-Methoxyphenyl)piperazin-1-yl]ethyl}-1,5,6,7-tetrahydroindol-4-one |
| 8 | 1-{3-[4-(4-Methoxyphenyl)piperazin-1-yl]propyl}-1,5,6,7-tetrahydroindol-4-one |
| 9 | 1-{4-[4-(4-Methoxyphenyl)piperazin-1-yl]butyl}-1,5,6,7-tetrahydroindol-4-one |
| 10 | 1-{2-[4-(2-Fluorophenyl)piperazin-1-yl]ethyl}-1,5,6,7-tetrahydroindol-4-one |
| 11 | 1-{3-[4-(2-Fluorophenyl)piperazin-1-yl]propyl}-1,5,6,7-tetrahydroindol-4-one |
| 12 | 1-{4-[4-(2-Fluorophenyl)piperazin-1-yl]butyl}-1,5,6,7-tetrahydroindol-4-one |
| 13 | 1-{2-[4-(4-Trifluoromethylphenyl)piperazin-1-yl]ethyl}-1,5,6,7-tetrahydroindol-4-one |
| 14 | 1-{3-[4-(4-Trifluoromethylphenyl)piperazin-1-yl]propyl}-1,5,6,7-tetrahydroindol-4-one |
| 15 | 1-{4-[4-(4-Trifluoromethylphenyl)piperazin-1-yl]butyl}-1,5,6,7-tetrahydroindol-4-one |
| 16 | 1-{2-[4-(4-Bromophenyl)piperazin-1-yl]ethyl}-1,5,6,7-tetrahydroindol-4-one |
| 17 | 1-{3-[4-(4-Bromophenyl)piperazin-1-yl]propyl}-1,5,6,7-tetrahydroindol-4-one |
| 18 | 1-{5-[4-(4-Bromophenyl)piperazin-1-yl]pentyl}-1,5,6,7-tetrahydroindol-4-one |
| 19 | 1-{2-[4-p-Tolylpiperazin-1-yl]ethyl}-1,5,6,7-tetrahydroindol-4-one |
| 20 | 1-{3-[4-p-Tolylpiperazin-1-yl]propyl}-1,5,6,7-tetrahydroindol-4-one |
| 21 | 1-{4-[4-p-Tolylpiperazin-1-yl]butyl}-1,5,6,7-tetrahydroindol-4-one |
| 22 | 1-{2-[4-(2,3-Dimethylphenyl)piperazin-1-yl]ethyl}-1,5,6,7-tetrahydroindol-4-one |
| 23 | 1-{3-[4-(2,3-Dimethylphenyl)piperazin-1-yl]propyl}-1,5,6,7-tetrahydroindol-4-one |
| 24 | 1-{4-[4-(2,3-Dimethylphenyl)piperazin-1-yl]butyl}-1,5,6,7-tetrahydroindol-4-one |
| 25 | 1-{2-[4-(3,4-Dichlorophenyl)piperazin-1-yl]ethyl}-1,5,6,7-tetrahydroindol-4-one |
| 26 | 1-{3-[4-(3,4-Dichlorophenyl)piperazin-1-yl]propyl}-1,5,6,7-tetrahydroindol-4-one |
| 27 | 1-{4-[4-(3,4-Dichlorophenyl)piperazin-1-yl]butyl}-1,5,6,7-tetrahydroindol-4-one |
| 28 | 1-{2-[4-(3,4-Difluorophenyl)piperazin-1-yl]ethyl}-1,5,6,7-tetrahydroindol-4-one |
| 29 | 1-{3-[4-(3,4-Difluorophenyl)piperazin-1-yl]propyl}-1,5,6,7-tetrahydroindol-4-one |
| 30 | 1-{4-[4-(3,4-Difluorophenyl)piperazin-1-yl]butyl}-1,5,6,7-tetrahydroindol-4-one |
| 31 | 1-{2-[4-(3,4-Dimethylphenyl)piperazin-1-yl]ethyl}-1,5,6,7-tetrahydroindol-4-one |
| 32 | 1-{3-[4-(3,4-Dimethylphenyl)piperazin-1-yl]propyl}-1,5,6,7-tetrahydroindol-4-one |
| 33 | 1-{4-[4-(3,4-Dimethylphenyl)piperazin-1-yl]butyl}-1,5,6,7-tetrahydroindol-4-one |
| 34 | 1-{2-[4-(2,3-Dichlorophenyl)piperazin-1-yl]ethyl}-1,5,6,7-tetrahydroindol-4-one |
| 35 | 1-{3-[4-(2,3-Dichlorophenyl)piperazin-1-yl]propyl}-1,5,6,7-tetrahydroindol-4-one |
| 36 | 1-{4-[4-(2,3-Dichlorophenyl)piperazin-1-yl]butyl}-1,5,6,7-tetrahydroindol-4-one |
| 37 | 1-[2-(4-(2-Naphthyl)piperazin-1-yl)ethyl]-1,5,6,7-tetrahydroindol-4-one |
| 38 | 1-[3-(4-(2-Naphthyl)piperazin-1-yl)propyl]-1,5,6,7-tetrahydroindol-4-one |
| 39 | 1-[4-(4-(2-Naphthyl)piperazin-1-yl)butyl]-1,5,6,7-tetrahydroindol-4-one |
| 40 | 1-{2-[4-(2,3-Dihydrobenzo[1,4]dioxin-6-yl)piperazin-1-yl]ethyl}-1,5,6,7-tetrahydroindol-4-one |
| 41 | 1-{3-[4-(2,3-Dihydrobenzo[1,4]dioxin-6-yl)piperazin-1-yl]propyl}-1,5,6,7-tetrahydroindol-4-one |
| 42 | 1-{4-[4-(2,3-Dihydrobenzo[1,4]dioxin-6-yl)piperazin-1-yl]butyl}-1,5,6,7-tetrahydroindol-4-one |
| 43 | 1-{2-[4-(2,4-Dichlorophenyl)piperazin-1-yl]ethyl}-1,5,6,7-tetrahydroindol-4-one |
| 44 | 1-{3-[4-(2,4-Dichlorophenyl)piperazin-1-yl]propyl}-1,5,6,7-tetrahydroindol-4-one |
| 45 | 1-{4-[4-(2,4-Dichlorophenyl)piperazin-1-yl]butyl}-1,5,6,7-tetrahydroindol-4-one |
| 46 | 1-{2-[4-(2,4-Difluorophenyl)piperazin-1-yl]ethyl}-1,5,6,7-tetrahydroindol-4-one |
| 47 | 1-{3-[4-(2,4-Difluorophenyl)piperazin-1-yl]propyl}-1,5,6,7-tetrahydroindol-4-one |
| 48 | 1-{4-[4-(2,4-Difluorophenyl)piperazin-1-yl]butyl}-1,5,6,7-tetrahydroindol-4-one |
| 49 | 1-{2-[4-(2,4-Dimethylphenyl)piperazin-1-yl]ethyl}-1,5,6,7-tetrahydroindol-4-one |
| 50 | 1-{3-[4-(2,4-Dimethylphenyl)piperazin-1-yl]propyl}-1,5,6,7-tetrahydroindol-4-one |
| 51 | 1-{4-[4-(2,4-Dimethylphenyl)piperazin-1-yl]butyl}-1,5,6,7-tetrahydroindol-4-one |
| 52 | 1-{2-[4-(5-Bromopyrimidin-2-yl)piperazin-1-yl]ethyl}-1,5,6,7-tetrahydroindol-4-one |
| 53 | 1-{3-[4-(5-Bromopyrimidin-2-yl)piperazin-1-yl]propyl}-1,5,6,7-tetrahydroindol-4-one |
| 54 | 1-{4-[4-(5-Bromopyrimidin-2-yl)piperazin-1-yl]butyl}-1,5,6,7-tetrahydroindol-4-one |
| 55 | 1-{2-[4-(2,3,4-Trichlorophenyl)piperazin-1-yl]ethyl}-1,5,6,7-tetrahydroindol-4-one |
| 56 | 1-{3-[4-(2,3,4-Trichlorophenyl)piperazin-1-yl]propyl}-1,5,6,7-tetrahydroindol-4-one |
| 57 | 1-{4-[4-(2,3,4-Trichlorophenyl)piperazin-1-yl]butyl}-1,5,6,7-tetrahydroindol-4-one |
| 58 | 1-{2-[4-(2,3,4-Trifluorophenyl)piperazin-1-yl]ethyl}-1,5,6,7-tetrahydroindol-4-one |
| 59 | 1-{3-[4-(2,3,4-Trifluorophenyl)piperazin-1-yl]propyl}-1,5,6,7-tetrahydroindol-4-one |
| 60 | 1-{4-[4-(2,3,4-Trifluorophenyl)piperazin-1-yl]butyl}-1,5,6,7-tetrahydroindol-4-one |
| 61 | 1-{2-[4-(3-Chloro-4-fluorophenyl)piperazin-1-yl]ethyl}-1,5,6,7-tetrahydroindol-4-one |
| 62 | 1-{3-[4-(3-Chloro-4-fluorophenyl)piperazin-1-yl]propyl}-1,5,6,7-tetrahydroindol-4-one |
| 63 | 1-{4-[4-(3-Chloro-4-fluorophenyl)piperazin-1-yl]butyl}-1,5,6,7-tetrahydroindol-4-one |
| 64 | 1-{5-[4-(3-Chloro-4-fluorophenyl)piperazin-1-yl]pentyl}-1,5,6,7-tetrahydroindol-4-one |
| 65 | 1-{2-[4-(4-Fluoro-3-trifluoromethylphenyl)piperazin-1-yl]ethyl}-1,5,6,7-tetrahydroindol-4-one |
| 66 | 1-{3-[4-(4-Fluoro-3-trifluoromethylphenyl)piperazin-1-yl]propyl}-1,5,6,7-tetrahydroindol-4-one |
| 67 | 1-{4-[4-(4-Fluoro-3-trifluoromethylphenyl)piperazin-1-yl]butyl}-1,5,6,7-tetrahydroindol-4-one |
| 68 | 1-{2-[4-(4-Chloro-2-methoxyphenyl)piperazin-1-yl]ethyl}-1,5,6,7-tetrahydroindol-4-one |
| 69 | 1-{3-[4-(4-Chloro-2-methoxyphenyl)piperazin-1-yl]propyl}-1,5,6,7-tetrahydroindol-4-one |
| 70 | 1-{4-[4-(4-Chloro-2-methoxyphenyl)piperazin-1-yl]butyl}-1,5,6,7-tetrahydroindol-4-one |
| 71 | 1-{2-[4-(4-Chloro-3-trifluoromethylphenyl)piperazin-1-yl]ethyl}-1,5,6,7-tetrahydroindol-4-one |
| 72 | 1-{3-[4-(4-Chloro-3-trifluoromethylphenyl)piperazin-1-yl]propyl}-1,5,6,7-tetrahydroindol-4-one |
| 73 | 1-{4-[4-(4-Chloro-3-trifluoromethylphenyl)piperazin-1-yl]butyl}-1,5,6,7-tetrahydroindol-4-one |
| 74 | 1-{5-[4-(4-Chloro-3-trifluoromethylphenyl)piperazin-1-yl]pentyl}-1,5,6,7-tetrahydroindol-4-one |
| 75 | 1-{2-[4-(6-Chloroquinolin-4-yl)piperazin-1-yl]ethyl}-1,5,6,7-tetrahydroindol-4-one |
| 76 | 1-{3-[4-(6-Chloroquinolin-4-yl)piperazin-1-yl]propyl}-1,5,6,7-tetrahydroindol-4-one |
| 77 | 1-{4-[4-(6-Chloroquinolin-4-yl)piperazin-1-yl]butyl}-1,5,6,7-tetrahydroindol-4-one |
| 78 | 1-{2-[4-(Thieno[3,2-d]pyrimidin-4-yl)piperazin-1-yl]ethyl}-1,5,6,7-tetrahydroindol-4-one |
| 79 | 1-{3-[4-(Thieno[3,2-d]pyrimidin-4-yl)piperazin-1-yl]propyl}-1,5,6,7-tetrahydroindol-4-one |
| 80 | 1-{4-[4-(Thieno[3,2-d]pyrimidin-4-yl)piperazin-1-yl]butyl}-1,5,6,7-tetrahydroindol-4-one |
| 81 | 1-{2-[4-(4-Chloronaphthalen-1-yl)piperazin-1-yl]ethyl}-1,5,6,7-tetrahydroindol-4-one |
| 82 | 1-{3-[4-(4-Chloronaphthalen-1-yl)piperazin-1-yl]propyl}-1,5,6,7-tetrahydroindol-4-one |
| 83 | 1-{4-[4-(4-Chloronaphthalen-1-yl)piperazin-1-yl]butyl}-1,5,6,7-tetrahydroindol-4-one |
| 84 | 1-{2-[4-(Furo[3,2-c]pyridine-4-yl)piperazin-1-yl]ethyl}-1,5,6,7-tetrahydroindol-4-one |
| 85 | 1-{3-[4-(Furo[3,2-c]pyridine-4-yl)piperazin-1-yl]propyl}-1,5,6,7-tetrahydroindol-4-one |
| 86 | 1-{4-[4-(Furo[3,2-c]pyridine-4-yl)piperazin-1-yl]butyl}-1,5,6,7-tetrahydroindol-4-one |
| 87 | 1-{2-[4-(4-Chloro-2-fluorophenyl)piperazin-1-yl]ethyl}-1,5,6,7-tetrahydroindol-4-one |
| 88 | 1-{3-[4-(4-Chloro-2-fluorophenyl)piperazin-1-yl]propyl}-1,5,6,7-tetrahydroindol-4-one |
| 89 | 1-{4-[4-(4-Chloro-2-fluorophenyl)piperazin-1-yl]butyl}-1,5,6,7-tetrahydroindol-4-one |

### Compound Properties

Preferred compounds of the present invention have a logP of from about 1 to about 4 to enhance bioavailability and, when desired, central nervous system (CNS) penetration. Using this guideline, one of ordinary skill in the art can choose the appropriate arylpiperazine moieties to use in combination with a particular A moiety in order to ensure the bioavailability and CNS penetration of a compound of the present invention. For example, if a highly hydrophobic A moiety is chosen, with particularly hydrophobic substituents, then a more hydrophilic arylpiperazine moiety can be used.

A number of the present compounds are optically active, owing to the presence of chiral carbons or other centers of asymmetry. All of the possible enantiomers or diastereoisomers of such compounds are included herein unless otherwise indicated despite possible differences in activity.

In general, the present compounds also include salts and prodrug esters of the compounds described herein. It is well known that organic compounds, including substituted tetrahydroindolones, arylpiperazines and other components of the present compounds, have multiple groups that can accept or donate protons, depending upon the pH of the solution in which they are present. These groups include carboxyl groups, hydroxyl groups, amino groups, sulfonic acid groups, and other groups known to be involved in acid-base reactions. The recitation of a compound in the present application includes such salt forms as occur at physiological pH or at the pH of a pharmaceutical composition unless specifically excluded.

Similarly, prodrug esters can be formed by reaction of either a carboxyl or a hydroxyl group on the compound with either an acid or an alcohol to form an ester. Typically, the acid or alcohol includes an alkyl group such as methyl, ethyl, propyl, isopropyl, butyl, isobutyl, and tertiary butyl. These groups can be substituted with substituents such as hydroxy, halo, or other substituents. Such prodrugs are well known in the art. The prodrug is converted into the active compound by hydrolysis of the ester linkage, typically by intracellular enzymes. Other suitable groups that can be used to form prodrug esters are well known in the art.

### SYNTHESIS EXAMPLES

The following representative methods for synthesizing exemplary compounds used in the present invention are merely intended as examples. Persons having ordinary skill in the art of medicinal and/or organic chemistry will understand that other starting materials, intermediates, and reaction conditions are possible. Furthermore, it is understood that various salts and esters of these compounds are also easily made and that these salts and esters can have biological activity similar or equivalent to the parent compound. Generally, such salts have halides or organic acids as anion counterions. However, other anions can be used and are considered within the scope of the present invention.

### Example 1

### Synthesis of 1-{2-[4-(3-Trifluoromethylphenyl)piperazin-1-yl]ethyl}-1,5,6,-7-tetrahydroindol-4-one

This example demonstrates a method of preparing 1-{2-[4-(3-Trifluoromethylphenyl)piperazin-1-yl]ethyl}-1,5,6,7-tetrahydroindol-4-one by a two step procedure. Generally, the arylpiperazine moieties are prepared first, then the arylpiperazine molecules are reacted with tetrahydroindolones.

### Step 1: Preparation of 1-(2-Chloroethyl)-4-(3-trifluoromethylphenyl)piperazine

To a 100 mL flask was added 4-(3-trifluoromethylphenyl)piperazine HCl (5035 mg, 18.88 mmol) and 60 mL dichloromethane. 1-Bromo-2-chloroethane (1730 μL, 20.78 mmol, 1.10 eq) was added, then triethylamine (5.25 mL, 37.7 mmol, 2.00 eq). The solution was refluxed for 9 hours, then cooled to 25° C. 100 mL of hexane was then added, and the resulting suspension was vacuum filtered. The filtrate was concentrated in vacuo and purified by column chromatography using dichloromethane as eluant resulting in an oil of 1-(2-chloroethyl)-4-(3-trifluoromethylphenyl)piperazine.

### Step 2: Preparation of 1-{2-[4-(3-Trifluoromethylphenyl)piperazin-1-yl]ethyl}-1,5,6,7-tetrahydroindol-4-one

Sodium hydride (60% in oil) (85 mg, 2.1 mmol, 1.8 eq.) was added to a 10 mL pear-shaped flask. The solid was rinsed twice with 2 mL hexane to remove oil, then 3 mL anhydrous N,N-dimethylformamide (DMF) was added. 1,5,6,7-Tetrahydroindol-4-one (186.7 mg, 1.38 mmol, 1.159 eq.) was added slowly, with stirring and hydrogen evolved. The walls of the flask were washed with an additional 1 mL of anhydrous DMF. 1-(2-Chloroethyl)-4-(3-trifluoromethylphenyl)piperazine (349.00 mg, 1.19 mmol, 1.000 eq) was added as a solution in 2 mL DMF, and the mixture was stirred under nitrogen at 25 C for 8 hours. The resulting mixture was acidified with 1N HCl to pH 6, and extracted with dichloromethane. The organic layer was washed four times with 25 mL water, dried over sodium sulfate and concentrated in vacuo to an oil which was purified by column chromatography using 5% methanol in dichloromethane, as eluant resulting in the title compound as an oil. The oil was dissolved in 5 mL of 50% dichloromethane in hexanes.

A solution of 4N HCl in dioxane (200 μL) was added and the mixture stirred for 30 minutes followed by vacuum filtration of the suspension. A white powder of the product HCl salt was recovered.

### Example 2

### Synthesis of 1-{3-[4-(3-Trifluoromethylphenyl)piperazin-1-yl]propyl}-1,5,6,7-tetrahydroindol-4-one

### Step 1: Preparation of 1-(3-Chloropropyl)-4-(3-trifluoromethylphenyl)piperazine

To a 100 mL flask was added 1-(3-trifluoromethylphenyl)piperazine HCl (5035 mg, 18.88 mmol) and 60 mL dichloromethane. 1-Bromo-3-chloropropane (1730 □L, 20.78 mmol, 1.10 eq) was added, then triethylamine (5.25 mL, 37.7 mmol, 2.00 eq). The solution was refluxed for 9 hours, then cooled to 25° C. 100 mL of hexane was then added, and the resulting suspension was vacuum filtered. The filtrate was concentrated in vacuo and purified by column chromatography using dichloromethane as eluant resulting in an oil of 1-(3-chloropropyl)-4-(3-trifluoromethylphenyl)piperazine.

### Step 2: Preparation of 1-{2-[4-(3-Trifluoromethylphenyl)piperazin-1-yl]propyl}-1,5,6,7-tetrahydroindol-4-one

The compound is synthesized by reacting the 1-(3-chloropropyl)-4-(3-trifluoromethylphenyl)piperazine with 1,5,6,7-tetrahydroindol-4-one using step 2 of Example 1.

### Example 3

### Synthesis of 1-{3-[4-(3-Chlorophenyl)piperazine-1-yl]propyl}-1,5,6,7-tetrahydroindol-4-one

Since 1-(3-Chloropropyl)-4-(3-chlorophenyl)piperazine HCl is commercially available, step one was omitted.

To a solution of 1,5,6,7-tetrahydroindol-4-one (135 mg, 1.0 mmol) in 5 mL dimethylsulfoxide was added powdered sodium hydroxide (84 mg, 2.1 mmol) and the solution stirred for 15 minutes at 25° C. 1-(3-Chloropropyl)-4-(3-chlorophenyl)piperazine HCl (310 mg, 1.0 mmol) was then added and stirring continued overnight. Upon completion, by thin-layer chromatography (TLC), the reaction was partitioned between 50 mL each of dichloromethane and water then separated. The water layer was extracted with 50 mL more of dichloromethane and the combined organic layers washed with brine, dried with sodium sulfate, and concentrated in vacuo to dryness. The crude product was purified via flash chromatography eluting with an ethyl acetate and dichloromethane mixture resulting in the title compound as an oil. The oil was dissolved in 5 mL of 50% dichloromethane in hexanes. A solution of 4N HCl in dioxane (200 μL) was added and the mixture stirred for 30 minutes followed by vacuum filtration of the suspension. A white powder of the product HCl salt was recovered.

### Example 4

### Synthesis of 1-{3-[4-(2-Methoxyphenyl)piperazine-1-yl]propyl}-1,5,6,7-tetrahydroindol-4-one

### Step 1: Preparation of 1-(3-Chloropropyl)-4-(2-methoxyphenyl)piperazine

The 1-(3-Chloropropyl)-4-(3-trifluoromethylphenyl)piperazine is prepared by the same method as disclosed in step 1 of example 2 employing 1-(2-Methoxyphenyl)piperazine HCl instead.

### Step 2: Preparation of 1-{3-[4-(2-Methoxyphenyl)piperazine-1-yl]propyl}-1,5,6,7-tetrahydroindol-4-one

The compound is prepared by the same method as disclosed in step 2 of example 3.

### Example 5

### Synthesis of 1-{3-[4-(2-Pyrimidyl)piperazine-1-yl]propyl}-1,5,6,7-tetrahydroindol-4-one

### Step 1: Preparation of 1-(3-Chloropropyl)-4-(2-pyrimidyl)piperazine

The compound is prepared by the same method as disclosed in step 1 of example 2 employing 1-(2-Pyrimidyl)piperazine.2HCl instead.

### Step 2: Preparation of 1-{3-[4-(2-Pyrimidyl)piperazine-1-yl]propyl}-1,5,6,7-tetrahydroindol-4-one

The compound is prepared by the same method as disclosed in step 2 of Example 3.

### Example 6

### Synthesis of 1-{2-[4-(3-Chlorophenyl)piperazin-1-yl]ethyl}-1,5,6,7-tetrahydroindol-4-one (Compound A)

### Step 1: Preparation of 1-(2-Chloroethyl)-4-(3-chlorophenyl)piperazine

A mixture of (3-chlorophenyl)piperazine HCl (51.5 mmol) and powdered sodium hydroxide (103 mmol) in DMSO (75 mL) was treated with 2-bromo-1-chloroethane (77.2 mmol) and stirred at ambient temperature for 4 hours. The reaction was poured into ice cold water (200 mL) and stirred for 0.5 hours. A solid mass formed and was separated by decanting the water. The aqueous layer was extracted with dichloromethane (100 mL). The solid mass was dissolved with dichloromethane (100 mL) and the combined organics were dried with sodium sulfate, filtered and the solvent removed under vacuum. Flash chromatography (chloroform:acetone 50:1 to 20:1) yielded an oil (7.95 g) as the titled compound.

### Step 2: 1-{2-[4-(3-Chlorophenyl)piperazin-1-yl]ethyl}-1,5,6,7-tetrahydroindol-4-one

To a solution of 1,5,6,7-tetrahyroindol-4-one (51.5 mmol) in DMSO (60 mL) was added powdered sodium hydroxide (53.9 mmol) and the mixture was stirred at ambient temperature for 0.5 hours. 1-(2-chloroethyl)-4-(3-chlorophenyl)piperazine (49.0 mmol) was then added as a solution in DMSO (20 mL) and the resulting mixture stirred at ambient temperature for 24 hours then heated to approximately 60° C. for 2 hours, after which time TLC (ethyl acetate:dichloromethane 1:1) showed complete reaction. The reaction was poured into ice cold water (300 mL) and stirred for 0.5 hours. A solid mass formed and was separated by decanting the water. The aqueous layer was extracted with dichloromethane (100 mL). The solid mass was dissolved with dichloromethane (100 mL) and the combined organics were dried with sodium sulfate and the solvent removed under vacuum. The resulting sludge was triturated with hexanes (100 mL) for 2 hours and the suspension vacuum filtered and washed with hexanes. The obtained solid was dried under vacuum resulting in a tan powder (14.57 g) as the titled compound.

### Example 7

### Synthesis of 1-{2-[4-(2-Methoxyphenyl)piperazin-1-yl]ethyl}-1,5,6,7-tetrahydroindol-4-one

### Step 1: Preparation of 1-(2-Chloroethyl)-4-(2-methoxyphenyl)piperazine

A mixture of 1-(2-methoxyphenyl)piperazine HCl (52.5 mmol) and powdered sodium hydroxide (105 mmol) in DMSO (40 mL), was stirred at ambient temperature. After 0.5 hours, 1-bromo-2-chloroethane (78.8 mmol) was added to the solution and left to stir for 4 hours. The reaction was monitored by TLC (ethyl acetate:dichloromethane 1:4), upon completion, the mixture was poured into 200 mL of ice water and the product was extracted with dichloromethane twice, dried with sodium sulfate, and solvent was removed under vacuum. Flash chromatography (ethyl acetate:dichloromethane, 1:5 yielded an oil of the title compound (7.30 g).

### Step 2: Preparation of 1-{2-[4-(2-Methoxyphenyl)piperazin-1-yl]ethyl}-1,5,6,7-tetrahydroindol-4-one

A mixture of 1,5,6,7-tetrahyroindol-4-one (30.1 mmol) and powdered sodium hydroxide (31.6 mmol) in DMSO (15 mL) was heated for 0.5 h, and then treated with a solution of 1-(2-chloroethyl)-4-(2-methoxyphenyl)piperazine (7.30 g) in DMSO (30 mL) dropwise. The reaction was left under heat and was monitored by TLC (ethyl acetate:dichloromethane, 1:1). After completion (−8 hours), the reaction mixture was poured into ice water (300 mL) and extracted with dichloromethane twice, dried with sodium sulfate and the solvent removed under vacuum. Flash chromatography (ethyl acetate: dichloromethane, 1:4) yielded an oil, (7.25 g).

### Example 8

### Synthesis of 1-{4-[4-(3-Trifluoromethylphenyl)piperazin-1-yl]butyl}-1,5,6,-7-tetrahydroindol-4-one

### Step 1: Synthesis of 1-(4-Chlorobutyl)-1,5,6,7-tetrahydroindol-4-one

To a solution of 1,5,6,7-tetrahydroindol-4-one (10.0 g, 74.0 mmol) in acetone (300 mL) was added powdered sodium hydroxide (3.26 g, 81.4 mmol) and the mixture stirred at ambient temperature for 0.25 hours. 1-Bromo-4-chlorobutane (9.38 mL, 81.4 mmol) was then added and the resulting mixture stirred at ambient temperature for 7 hours after which time TLC (ethyl acetate:dichloromethane 1:1) showed complete reaction. The reaction was gravity filtered to remove salts, and the filtrate concentrated to dryness under vacuum. The resulting residue was dissolved in dichloromethane (200 mL) and gravity filtered again to remove more salts. The filtrate was then washed with water, dried with sodium sulfate, filtered and the solvent removed under vacuum to yield an oil. Flash chromatography using 6 in. of silica gel in a 5.5 cm column eluting with 1:1 followed by 2:1 ethyl acetate:hexane on half of the residue yielded 9.0 g of an oil which contained ˜6.0 g of pure product (72%) and ˜3.0 g of acetone aldol condensation product (4-hydroxy-4-methyl-2-pentanone). The oil was taken to the next step without further purification.

### Step 2: Synthesis of 1-{4-[4-(3-Trifluoromethylphenyl)piperazin-1-yl]butyl}-1,5,6,7-tetrahydroindol-4-one

A mixture of 1-(4-Chlorobutyl)-1,5,6,7-tetrahydroindol-4-one (6.0 g, 26.6 mmol, as a mixture with 3.0 g of 4-hydroxy-4-methyl-2-pentanone) and sodium iodide (4.38 g, 29.2 mmol) in acetonitrile (100 mL) was heated at reflux for 6 hours. (3-Trifluoromethylphenyl)piperazine (5.81 g, 25.2 mmol) and potassium carbonate (3.67 g, 26.6 mmol) was then added and reflux continued for 16 hours. TLC (ethyl acetate:dichloromethane 1:1) showed complete reaction. The reaction was poured into ice cold water (400 mL) and stirred for 0.5 hours. An oil separated out and was isolated from the mixture. The oil was dissolved with dichloromethane (150 mL), washed with water and brine, then dried with sodium sulfate, filtered and the solvent removed under vacuum to yield the title compound as an oil (9.7 g, 91.5%).

Preparation of Oxalate salt of 1-{4-[4-(3-Trifluoromethylphenyl)piperazin-1-yl]butyl}-1,5,6,7-tetrahydroindol-4-one. Dissolved compound (4.2 g) in hot ethyl acetate (150 mL), filtered solution hot to remove undissolved solid, and added a solution of oxalic acid (1.08 g, 1.2 eq) in methanol (10 mL) with stirring. A white precipitate formed immediately and the mixture was stirred for 0.5 hours to room temperature. Vacuum filtration and washing with ethyl acetate afforded an off-white powder upon drying (5.0 g, 98%). HPLC Purity was 98.9%.

### Example 9

### Synthesis of 1-{2-[4-(3,4-Dichlorophenyl)piperazin-1-yl]ethyl}-1,5,6,7-tetrahydroindol-4-one

### Step 1: Preparation of 1-(2-Chloroethyl)-4-(3,4-dichlorophenyl)piperazine

A mixture of (3,4-dichlorophenyl)piperazine (500 mg) and powdered sodium hydroxide (87 mg) in DMSO (5 mL) was treated with 2-bromo-1-chloroethane (387 mg) and stirred at ambient temperature for 16 hours. The reaction was poured into ice cold water (15 mL) and stirred for 0.5 hours. A solid mass formed and was separated by decanting the water. The aqueous layer was extracted with dichloromethane (5 mL). The solid mass was dissolved with dichloromethane (5 mL) and the combined organics were dried with sodium sulfate, filtered and the solvent removed under vacuum. Flash chromatography (dichloromethane:methanol 1:0 to 10:1) yielded an oil (230 mg) as the titled compound.

### Step 2: 1-{2-[4-(3,4-Dichlorophenyl)piperazin-1-yl]ethyl}-1,5,6,7-tetrahydroindol-4-one

To a solution of 1,5,6,7-tetrahyroindol-4-one (107 mg) in DMSO (2 mL) was added powdered sodium hydroxide (33 mg) and the mixture was stirred at ambient temperature for 0.5 hours. 1-(2-Chloroethyl)-4-(3,4-dichlorophenyl)piperazine (220 mg) from step 1 was then added as a solution in DMSO (2 mL) and the resulting mixture stirred at ambient temperature for 24 hours then heated to approximately 60° C. for 2 hours, after which time thin layer chromatography (TLC) (ethyl acetate:dichloromethane 1:1) showed complete reaction. The reaction was poured into ice cold water (15 mL) and stirred for 0.5 hours. A solid mass formed and was separated by decanting the water. The aqueous layer was extracted with dichloromethane (10 mL). The solid mass was dissolved with dichloromethane (5 mL) and the combined organics were dried with sodium sulfate and the solvent removed under vacuum to obtain an oil (250 mg) as the titled compound.

### Step 3: Preparation of Oxalate salt of 1-{2-[4-(3,4-Dichlorophenyl)piperazin-1-yl]ethyl}-1,5,6,7-tetrahydroindol-4-one

The compound from step 2 (250 mg) was dissolved in ethyl acetate (5 mL) using heat if required, and a solution of oxalic acid (57 mg) in acetone (0.5 mL) was added with stirring. A precipitate formed immediately and the mixture was stirred for 0.5 hours at room temperature. Vacuum filtration and washing with ethyl acetate afforded an off-white powder upon drying (220 mg).

The same 3-step procedure is used for all ethyl and propyl linkers.

### Example 10

### Synthesis of 1-{4-[4-(3,4-Dichlorophenyl)piperazin-1-yl]butyl}-1,5,6,7-tetrahydroindol-4-one

### Step 1: Synthesis of 1-(4-Chlorobutyl)-1,5,6,7-tetrahydroindol-4-one

To a solution of 1,5,6,7-tetrahydroindol-4-one (10.0 g) in DMSO (100 mL) was added powdered sodium hydroxide (3.26 g) and the mixture was stirred at ambient temperature for 0.25 hours. 1-Bromo-4-chlorobutane (9.38 mL) was then added and the resulting mixture stirred at ambient temperature for 7 hours after which time TLC (ethyl acetate:dichloromethane 1:1) showed complete reaction. The reaction was poured into ice cold water (250 mL) and stirred for 0.5 hours. An oil separated and was isolated with a separatory funnel. The aqueous layer was extracted with dichloromethane (50 mL). The oil was dissolved with dichloromethane (25 mL) and the combined organics were dried with sodium sulfate, filtered and the solvent removed under vacuum. Flash chromatography (ethyl acetate:hexane, 1:1 to 2:1) yielded an oil (6.0 g) as the titled compound.

### Step 2: Synthesis of 1-{4-[4-(3,4-Dichlorophenyl)piperazin-1-yl]butyl}-1,5,6,7-tetrahydroindol-4-one

A mixture of 1-(4-Chlorobutyl)-1,5,6,7-tetrahydroindol-4-one (600 mg) from step 1 and sodium iodide (438 mg) in acetonitrile (10 mL) was heated at reflux for 6 hours. (3,4-Dichlorophenyl)piperazine (581 mg) and potassium carbonate (367 mg) was then added and reflux continued for 16 h. TLC (ethyl acetate:dichloromethane 1:1) showed complete reaction. The reaction was poured into ice cold water (50 mL) and stirred for 0.5 hours. An oil separated out and was isolated from the mixture. The oil was dissolved with dichloromethane (15 mL), washed with water and brine, then dried with sodium sulfate, filtered and the solvent removed under vacuum to yield the title compound as an oil (970 mg).

### Step 3: Oxalate Salt Formation

Oxalate salt formation is done in the same manner as previously described.

The same 3-step procedure is used for all butyl linkers.

### Pharmaceutical Compositions

Another aspect of the present invention is a pharmaceutical composition that comprises: (1) an therapeutically effective amount of a compound according to the present invention as described above (including salts and esters thereof); and (2) a pharmaceutically acceptable excipient.

A pharmaceutically acceptable excipient, including carriers, can be chosen from those generally known in the art including, but not limited to, inert solid diluents, aqueous solutions, or non-toxic organic solvents, depending on the route of administration. If desired, these pharmaceutical formulations can also contain preservatives and stabilizing agents and the like, for example substances such as, but not limited to, pharmaceutically acceptable excipients selected from the group consisting of wetting or emulsifying agents, pH buffering agents, human serum albumin, antioxidants, preservatives, bacteriostatic agents, dextrose, sucrose, trehalose, maltose, lecithin, glycine, sorbic acid, propylene glycol, polyethylene glycol, protamine sulfate, sodium chloride, or potassium chloride, mineral oil, vegetable oils and combinations thereof. Those skilled in the art will appreciate that other carriers also can be used.

Liquid compositions can also contain liquid phase excipients either in addition to or to the exclusion of water. Examples of such additional liquid phases are glycerin, vegetable oils such as cottonseed oil, organic esters such as ethyl oleate, and water-oil emulsions.

Formulations suitable for parenteral administration, such as, for example, by intravenous, intramuscular, intradermal, and subcutaneous routes, include aqueous and non-aqueous isotonic sterile injection solutions. These can contain antioxidants, buffers, preservatives, bacteriostatic agents, and solutes that render the formulation isotonic with the blood of the particular recipient. Alternatively, these formulations can be aqueous or non-aqueous sterile suspensions that can include suspending agents, thickening agents, solubilizers, stabilizers, and preservatives. The pharmaceutical compositions of the present invention can be formulated for administration by intravenous infusion, oral, topical, intraperitoneal, intravesical, transdermal, intranasal, rectal, vaginal, intramuscular, intradermal, subcutaneous and intrathecal routes.

Formulations of compound suitable for use in methods according to the present invention can be presented in unit-dose or multi-dose sealed containers, in physical forms such as ampules or vials. The compositions can be made into aerosol formations (i.e., they can be “nebulized”) to be administered via inhalation. Aerosol formulations can be placed into pressurized acceptable propellants, such as dichloromethane, propane, or nitrogen. Other suitable propellants are known in the art.

### Preclinical Models and Clinical Evaluation

In order to screen for the most effective of the present compounds and pharmaceutical compositions and determine appropriate candidates for further development, as well as to determine appropriate dosages of such compounds and compositions for a human subject, preclinical animal models can be used. Exemplary animal models are set forth below. Preferably, a series of tests is performed in animal models to screen for activity in treating and/or preventing PTSD.

Compounds and compositions are preferably selected using a panel of pre-clinical tests. Preliminary screening tests can be used to determine appropriate dosages to test in follow-on models. Appropriately selected doses of compounds and compositions tested in this way can then be subjected to testing for efficacy in models that mimic certain aspects of PTSD and/or which reduce fear responses and/or the memory of fear associated with a triggering event. Preferred compounds and compositions will produce such effects at doses which do not significantly affect learning, the acquisition of memories, and memory recall not associated with severe traumatic events.

A. Models for Determining Appropriate Dosages

1. Neuromuscular Coordination Model (Rotarod)

This model can be used to determine the dose of a compound or composition at which unwanted side effects (muscle tone/motor coordination deficits) occur. Animals (C57 Mice) are placed on a rotarod treadmill (model V EE/85, Columbus Instruments, Columbus, Ohio) accelerating from 1 to 80 revolutions/4 minutes. All mice are given two control trials at least 12 hours before oral administration evaluation of compounds. Mice are tested on the rotarod 30 minutes after administration of compounds. The number of seconds each mouse remained on the rotarod is recorded.

Doses at which the coordination of an animal is decreased or at which its motor function is altered, such that the ability of the animal to remain on the rotarod is reduced, are determined. Doses below this are selected for further evaluation.

2. Spontaneous Activity Model (Locomotor Activity)

Ambulatory and non-ambulatory activity can be used to test spontaneous and drug-induced motor activity. The test can be used to profile the potential for a drug to induce hyperactivity or sedation.

In this model, Kinder Scientific photobeam activity monitors are used to record the ambulatory and non-ambulatory motor activity. The monitors track the photobeam breaks made by the animal that are used to calculate the number of ambulatory and fine (non-ambulatory) motor movements. A drug-induced increase in activity can indicate the potential for an adverse event such as hyperactivity. A drug-induced decrease in response can indicate the potential for an adverse event such as sedation. Doses at which no significant change in activity are recorded, and more preferably at which no change in activity are recorded, can be selected for further evaluation.

3. Potentiated Startle (Anxiety Model)

This model can be used to evaluate anxiolytic or anxiogenic effects of a candidate molecule. In this model, Hamilton-Kinder startle chambers can be used for conditioning sessions and for the production and recording of startle responses. A classical conditioning procedure is then used to produce potentiation of startle responses. On the first of 2 days, rats, preferably Long Evans rats, are placed into dark startle chambers having shock grids. Following a 5-minute acclimation period, each rat is administered a 1 mA electric shock (500 ms) preceded by a 5 second presentation of light (15 watt) which remains on for the duration of the shock. Ten presentations of the light and shock are given in each conditioning session.

The rats are then administered a test compound, after which startle testing sessions are conducted. A block of 10 consecutive presentations of acoustic startle stimuli (110 dB, non-light-paired) are presented at the beginning of the session in order to minimize the influences of the initial rapid phase of habituation to the stimulus. This is followed by 20 alternating trials of the noise alone or noise preceded by the light. Excluding the initial trial block, startle response amplitudes for each trial type (noise−alone vs. light+noise) are averaged for each rat across the entire test session.

Compounds and compositions appropriate development preferably do not result in either anxiogenic or anxioiytic activity.

2. Other Models

Other models that can be used to evaluate proper dosages of the present compounds and compositions include the Elevated Plus Maze model, which also evaluates the anxiogenic or anxiolytic activity of a candidate.

B. Learning and Memory Models

Appropriate doses of the present compounds and compositions can be tested in models of cognition, in particular of learning, memory acquisition, consolidation, and recall. Compounds and compositions which do not significantly adversely affect these functions in animal models, and which preferably have no effect or have a beneficial effect, are preferably selected as candidates for further evaluation.

1. Conditioned Avoidance Model

The Condition Avoidance Responding (CAR, active avoidance) model is a measure of cognitive and attention impairment. It evaluates the disruption of avoidance (increased latency) without disruption of escape (extrapyramidal motor function).

The training of C-57 mice consists of 20 trials with variable inter-trial intervals (trained to 80% Avoidance Criteria). After a one-minute acclimation period, the house light and an acoustic 90 decibel tone (conditioned stimuli) are presented. A response (crossing to dark compartment) within 5 seconds ends the trial and trial is recorded as avoidance response (CAR). If the mouse does not respond within 5 seconds, foot shock (0.8 mA) is presented, and the response (moving to the dark chamber) during the shock was recorded as an escape response. To avoid shock, animals learn to move from the lighted side of the − chamber to the dark side when the cue is presented (avoidance) or moved when the shock is administered (escape). Compounds that disrupt cognition in the CAR model can be excluded from candidate consideration.

2. Other Models

Other models include the Acquisition of Active Avoidance (Memory Acquisition/Retention) model, in which a result of no effect on memory acquisition/retention indicates that learning is not being impaired by a candidate and that the candidate can be further evaluated for development. The three Trial Passive Avoidance (Memory Acquisition/Retention) model can also be evaluated in order to determine whether a candidate molecule or composition affects on memory acquisition/retention/consolidation. Other learning models include the Morris Water Maze and the Amnesic Reversal model.

C. Fear Models

Screening of candidate compounds and compositions for activity in reducing fear responses and/or the memory of fear associated with a triggering event is preferably performed.

1. Passive Avoidance

The passive avoidance model is an example of a fear conditioning behavior. To perform this test, animals were trained and tested in a Kinder Scientific avoidance system consisting of a shuttle box with a shock grid floor. On day one, mice were introduced to the system by being allowed to move freely between a darkened side and a lighted side of the shuttle box for 3 minutes after a 1 minute acclimation in the dark. Day two (24 hours later) was similar to the first day except that animals received a 1.0 mA foot shock after crossing to the dark compartment. Vehicle or test compounds are administered subcutaneously 30 minutes prior to this training day. Twenty four hours after the training day, animals were put back in the shuttle box and after a minute acclimation period in the dark, the latency to cross from the lighted to the dark compartment (now with the shock off) was recorded.

2. Other Models

Other models include Three-Day Single Trial Passive Avoidance (Fear-Memory Recall), Results indicating a reduced magnitude of memory associated with fear in these models would indicate that tested compounds or compositions are candidates for further development.

D. Models of Aspects of ASD/PTSD

1. Prepulse Inhibition Model

The phencyclidine (PCP)-induced disruption of pre-pulse inhibition (PPI) has been used to predict efficacy in cognitive impairment and other disorders. In this test, a weaker auditory prestimulus (prepulse) inhibits the reaction of an test organism to a subsequent strong startle stimulus (pulse). Normal PPI functioning in mammals is critical in filtering out irrelevant sensory information, an inhibitory process that is affected in patients with PTSD. A reduction of the cognitive/attention impairment produced in the PPI model can be used as an efficacy measure for compound candidate selection.

For testing of PPI, male C57 mice can be assigned to five dose groups of eight animals per group, and vehicle or test compound can then be administered orally (PO) or subcutaneously (SC) 20 minutes prior to intraperitoneal (IP) administration of vehicle or PCP (5 mg/kg). Ten minutes following PCP administration, the mice are placed into Hamilton-Kinder startle chambers and evaluation of pre-pulse inhibition procedure is performed. Following a five-minute acclimatization period with background white noise (65 decibels), mice were exposed to five different trial types. Trials were presented ten-time search in a quasi-random order, with randomized 5 to 25 second inter-trial intervals. Trials were: stimulus only trial (120 decibel white noise, 50 ms stimulus); two different prepulse+pulse trials in which a 20 ms 5 decibel, or 10 decibel stimuli above a 65 decibel background preceded the 120 decibel pulse by 120 ms; a 10 decibel prepulse without a 120 decibel pulse; and a no stimulus trial, in which only the background noise was presented.

Compounds which are observed to reduce and/or reverse PCP-induced impairment can be selected as candidates for further evaluation. Such reduction is also indicative of memory enhancement, which is also a positive indicator for further development.

2. Stress-Induced Motor Suppression (Emotional Response to Fear)

Stress-induced motor suppression in rodents is a measure of conditioned fear stress, an animal response relevant to the clinical manifestations of PSTD. Behavioral testing is carried out as previously described with minor modifications [Kamei et al., “Activation of both dopamine D1 and D2 receptors necessary for amelioration of conditioned fear stress,” European Journal of Pharmacology, 273:229-233 (1995)] using male C57 mice. Training and testing are conducted on consecutive days using Kinder Scientific photobeam activity monitors with a shock grid floors. By tracking the photobeam breaks, activity monitors record the time spent immobile, as well as ambulatory and non-ambulatory motor activity. Rodents receive a SC or vehicle administration approximately 30 minutes prior to the training session. Training consists of a 3 minute period in which animals moved freely through the activity chamber, followed by a 5 second 1.0 mA foot shock. Animals then remain in the system for an additional minute before removal from the chamber. Twenty four hours later, animals are returned to the same activity chamber, but no foot shock is administered, and fine movements, ambulatory movements, and time spent immobile are recorded for 4 minutes. Rodents that develop a stronger association of the foot shock and the activity chamber on the previous day are expected to spend more time immobile on testing day.

3. Other Models

Other models include the Reversal of Amphetamine-Induced Hyper-Locomotion model, in which reversal of amphetamine-induced hyper-locomotion indicates that a candidate molecule or composition is affecting dopamine receptors, which are implicated in PTSD. Damage to or shrinking of the hippocampus has also been implicated in PTSD. Therefore, evaluation of the potentiation of neurite outgrowth, using W28-Neuro2a cells for example, can be examined in order to evaluate the appropriateness of a candidate for further development.

E. Clinical Development

Following the testing of candidate compounds and/or compositions in preclinical animal models, candidates for further development can be selected based on the criteria set forth above. One or more selected candidates having desirable preclinical profiles can then be subjected to clinical evaluation in human patients using methods known to those of skill in the art. Subjects for human clinical trials can be selected in the same manner as the selection of subjects appropriate for treatment with the present compounds and compositions, as set forth below.

### Acute Stress Disorder and Post-Traumatic Stress Disorder

In order to determine whether an individual is at risk of acquiring ASD and/or PTSD and is therefore a candidate for preventative treatment with the present compositions and/or compounds, the individual's current life situation can be assessed. If the individual is at risk of exposure to a terrifying event or situation in which grave physical harm (including death, either to the individual or someone else) may occur or be likely to occur, or in which grave physical harm may be threatened, then the individual is a candidate for treatment with the present compounds and/or compositions in order to prevent ASD and/or PTSD. Traumatic events that may trigger ASD and PTSD include violent personal assaults, natural or human-caused disasters, accidents, and military combat.

If an individual has experienced such a traumatic event but has not yet exhibited symptoms of ASD or PTSD, the individual can also be treated with the present compounds and/or compositions. Without limiting the generality of the present disclosure, it is believed that the present compounds modulate or interfere with the process by which memories are formed, reinforced, and/or associated with a emotional and/or physical response.

Preferably, an individual who has experienced a traumatic event but not yet exhibited symptoms of ASD or PTSD is treated within a week of exposure to such a traumatic event in order to effectively treat ASD and/or PTSD and prevent some or all of the symptomology associated with PTSD from occurring. More preferably, such an individual is treated within 24, 48, or 72 hours of exposure to the trauma, and even more preferably the individual is treated immediately following the event, i.e. within 1-6 hours of exposure to the traumatic event.

An individual who has already acquired ASD or PTSD can also be effectively treated with the present compounds and/or compositions. An individual who has acquired ASD or PTSD and who is therefore in need of treatment with the present compounds and/or compositions can be identified through the diagnosis of the individual by a skilled clinician, such as a psychologist or psychiatrist. Such a skilled clinician can make a diagnosis of PTSD by following the criteria contained in the DSM-IV, set forth in Table 2 below.

TABLE 2
DSM-IV Criteria for Post-Traumatic Stress Disorder

```
A. The person has been exposed to a traumatic event in which both of the following have been
present:
    (1) the person experienced, witnessed, or was confronted with an event or events that involved
actual or threatened death or serious injury, or a threat to the physical integrity of self or others.
    (2) the person's response involved intense fear, helplessness, or horror. Note: In children, this
may be expressed instead by disorganized or agitated behavior.
B. The traumatic event is persistently reexperienced in one (or more) of the following ways:
    (1) recurrent and intrusive distressing recollections of the event, including images, thoughts, or
perceptions. Note: In young children, repetitive play may occur in which themes or aspects of the
trauma are expressed.
    (2) recurrent distressing dreams of the event. Note: In children, there may be frightening
dreams without recognizable content.
    (3) acting or feeling as if the traumatic event were recurring (includes a sense of reliving the
experience, illusions, hallucinations, and dissociative flashback episodes, including those that
occur upon awakening or when intoxicated). Note: In young children, trauma-specific
reenactment may occur.
    (4) intense psychological distress at exposure to internal or external cues that symbolize or
resemble an aspect of the traumatic event.
    (5) physiological reactivity on exposure to internal or external cues that symbolize or resemble
an aspect of the traumatic event.
C. Persistent avoidance of stimuli associated with the trauma and numbing of general
responsiveness (not present before the trauma), as indicated by three (or more) of the following:
    (1) efforts to avoid thoughts, feelings, or conversations associated with the trauma.
    (2) efforts to avoid activities, places, or people that arouse recollections of the trauma.
    (3) inability to recall an important aspect of the trauma.
    (4) markedly diminished interest or participation in significant activities.
    (5) feeling of detachment or estrangement from others.
    (6) restricted range of affect (e.g., unable to have loving feelings).
    (7) sense of a foreshortened future (e.g., does not expect to have a career, marriage, children,
or a normal life span).
D. Persistent symptoms of increased arousal (not present before the trauma), as indicated by two
(or more) of the following:
    (1) difficulty falling or staying asleep.
    (2) irritability or outbursts of anger.
    (3) difficulty concentrating.
    (4) hypervigilance.
    (5) exaggerated startle response.
E. Duration of the disturbance (symptoms in Criteria B, C, and D) is more than one month.
F. The disturbance causes clinically significant distress or impairment in social, occupational, or
other important areas of functioning.
```

If an individual exhibits the appropriate combination of symptoms indicating a diagnosis of PTSD as outlined in Table 2, then that individual can be treated with the present compounds and/or compositions. In order to arrive at a diagnosis of PTSD, the patient's symptoms generally must significantly disrupt normal activities and last for more than one month. Diagnosis of another psychiatric disorder, such as depression, alcohol and drug abuse, or other anxiety disorder, may aid in diagnosis, as approximately 80 percent of patients with PTSD also have at least one other psychiatric disorder.

### Treatment of Acute Stress Disorder and Post-Traumatic Stress Disorder

Both ASD and PTSD can be prevented or treated by administering therapeutically effective amounts of one or more of the present compounds and/or pharmaceutical compositions to a patient in need thereof. The present compounds and/or compositions are administered to a patient in a quantity sufficient to treat or prevent the symptoms and/or the underlying etiology associated with ASD or PTSD in the patient. The present compounds can also be administered in combination with other agents known to be useful in the treatment of PTSD, such as paroxetine and sertraline, either in physical combination or in combined therapy through the administration of the present compounds and agents in succession (in any order).

Administration of the present compounds and compositions can begin immediately following exposure to a traumatic event, preferably within the first week following the traumatic event, and more preferably within the first 24-72 hours. Administration of the compositions and compounds can alternatively begin prior to an anticipated traumatic event (such as impending combat), in order to prevent or reduce the severity of subsequent ASD and/or PTSD. The present compounds and compositions can also be administered following a subject's experience of symptoms of ASD and/or PTSD, such as during either the acute, chronic, or delayed-onset phase. The present invention thus includes the use of the present compounds and/or a pharmaceutical composition comprising such compounds to prevent and/or treat ASD or PTSD.

Depending upon the particular needs of the individual subject involved, the present compounds can be administered in various doses to provide effective treatments for PTSD. Factors such as the activity of the selected compound, half life of the compound, the physiological characteristics of the subject, the extent or nature of the subject's disease or condition, and the method of administration will determine what constitutes an effective amount of the selected compounds. Generally, initial doses will be modified to determine the optimum dosage for treatment of the particular subject. The compounds can be administered using a number of different routes including oral administration, topical administration, transdermal administration, intraperitoneal injection, or intravenous injection directly into the bloodstream. Effective amounts of the compounds can also be administered through injection into the cerebrospinal fluid or infusion directly into the brain, if desired.

An effective amount of any embodiment of the present invention is determined using methods known to pharmacologists and clinicians having ordinary skill in the art. For example, the animal models described herein can be used to determine applicable dosages for a patient. As known to those of skill in the art, a very low dose of a compound, i.e. one found to be minimally toxic in animals (e.g., 1/10×LD10 in mice), can first be administered to a patient, and if that dose is found to be safe, the patient can be treated at a higher dose. A therapeutically effective amount of one of the present compounds for treating PTSD can then be determined by administering increasing amounts of such compound to a patient suffering from PTSD until such time as the patient's symptoms are observed or are reported by the patient to be diminished or eliminated.

In a preferred embodiment, the present compounds and compositions selected for use in treating or preventing PTSD for a particular subject or underlying condition have a therapeutic index of approximately 2 or greater. The therapeutic index is determined by dividing the dose at which adverse side effects occur by the dose at which efficacy for the condition is determined. A therapeutic index is preferably determined through the testing of a number of subjects. Another measure of therapeutic index is the lethal dose of a drug for 50% of a population (LD₅₀, in a pre-clinical model) divided by the minimum effective dose for 50% of the population (ED₅₀).

Blood levels of the present compounds can be determined using routine biological and chemical assays and these blood levels can be matched to the route of administration and half life of a selected compound. The blood level and route of administration giving the most desirable level of PTSD relief can then be used to establish a therapeutically effective amount of a pharmaceutical composition comprising one of the present compounds for preventing and/or treating PTSD.

Exemplary dosages in accordance with the teachings of the present invention for these compounds range from 0.0001 mg/kg to 60 mg/kg, though alternative dosages are contemplated as being within the scope of the present invention. Suitable dosages can be chosen by the treating physician by taking into account such factors as the size, weight, age, and sex of the patient, the physiological state of the patient, the severity of the condition for which the compound is being administered, the response to treatment, the type and quantity of other medications being given to the patient that might interact with the compound, either potentiating it or inhibiting it, and other pharmacokinetic considerations such as liver and kidney function.

### EXAMPLES

### Example 1

### Dose Selection

### Effects on Neuromuscular Coordination

Male Swiss Webster (CFW) mice were placed on a rotarod (model V EE/85, Columbus Instruments, Columbus, Ohio) accelerating from 1 to 80 revolutions/4 minutes. All mice were given two control trials at least 12 hours before administration of the present compounds for evaluation. Mice were tested on the rotarod 30 minutes after subcutaneous administration of compounds. The number of seconds each mouse remained on the rotarod was recorded. Compounds that decreased coordination or altered motor function reduced the ability of the animal to remain on the rotarod. The ED₅₀ for compound A in this test was 23 mg/kg.

### Example 2

### Further Dose Selection

The present compounds were tested in the conditioned avoidance response (CAR) model and in the Spontaneous Activity model described above. Compound A did not produce any effect when administered subcutaneously at doses of up to 10 mg/kg compared to vehicle treated mice in either the CAR model or the Spontaneous Activity model, suggesting that Compound A does not disrupt cognition or produce sedation.

### Example 3

### Prepulse Inhibition (PPI) Testing

Male C57 mice were assigned to five dose groups of eight animals per group, and vehicle or a test compound was administered orally or subcutaneously 20 minutes prior to intraperitoneal administration of vehicle or PCP (5 mg/kg). Ten minutes following PCP administration, the mice were placed into Kinder Scientific startle chambers (Kinder Scientific, Poway, Calif.) and pre-pulse inhibition was evaluated. Following a five-minute acclimatization period with background white noise (65 dB), mice were exposed to five different trial types. Trials were presented in a quasi-random order, with randomized 5 to 25 second inter-trial intervals. The five different trials (presented 10 times each were): stimulus only trial (120 dB white noise, 50 ms stimulus); two different prepulse+pulse trials in which a 20 ms 5 dB or 10 dB stimulus above a 65 dB background preceded the 120 dB pulse by 120 ms; a 10 dB prepulse without a 120 dB pulse; and a no stimulus trial, in which only the background noise was presented. Test results for the present compounds are shown in Table 3 below.

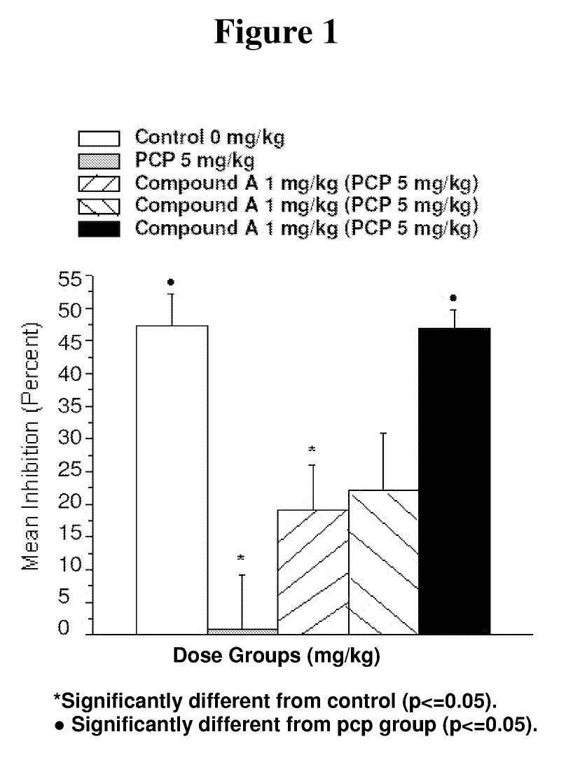

The pharmaceutical effect of the present compounds was found to reverse the PCP-induced disruption of prepulse inhibition of the startle response in male C57 mice in a dose-dependent manner, as shown in FIG. 1. FIG. 1 illustrates the results of tests involving compound A in the PPI model, and shows that at doses of 10 mg/kg inhibition was returned to control levels.

TABLE 3
Prepulse Inhibition Testing

| | Compound | Effective | Administration |
|---|---|---|---|
| Compound Name | Reference | Dose | Route |
| 1-{2-[4-(3-Chlorophenyl)piperazin-1- | A | 1.0 mg/kg | oral |
| yl]ethyl}-1,5,6,7-tetrahydroindol-4-one | | | |
| 1-{2-[4-(3-Chlorophenyl)piperazin-1- | A | 3.0 mg/kg | subcutaneous |
| yl]ethyl}-1,5,6,7-tetrahydroindol-4-one | | | |
| 1-{4-[4-(4-Fluorophenyl)piperazin-1-yl]butyl}- | B | 1.0 mg/kg | subcutaneous |
| 1,5,6,7-tetrahydroindol-4-one | | | |
| 1-{4-[4-(4-Bromophenyl)piperazine-1- | C | 3.0 mg/kg | subcutaneous |
| yl]butyl}-1,5,6,7-tetrahydroindol-4-one | | | |
| 1-{4-[4-(3-Trifluoromethylphenyl)piperazin-1- | D | 1.0 mg/kg | oral |
| yl]butyl}-1,5,6,7-tetrahydroindol-4-one | | | |
| 1-{4-[4-(3-Trifluoromethylphenyl)piperazin-1- | D | 0.3 mg/kg | subcutaneous |
| yl]butyl}-1,5,6,7-tetrahydroindol-4-one | | | |
| 1-{4-[4-(3,4-Dichlorophenyl)piperazin-1- | H | 6.0 mg/kg | oral |
| yl]butyl}-1,5,6,7-tetrahydroindol-4-one | | | |
| 1-{4-[4-(3,4-Dichlorophenyl)piperazin-1- | H | 1.0 mg/kg | subcutaneous |
| yl]butyl}-1,5,6,7-tetrahydroindol-4-one | | | |
| 1-{4-[4-(3-Chloro-4-fluorophenyl)piperazin-1- | I | 1.0 mg/kg | subcutaneous |
| yl]butyl}-1,5,6,7-tetrahydroindol-4-one | | | |
| 1-{4-[4-(2,4-Dichlorophenyl)piperazin-1- | J | 10.0 mg/kg | subcutaneous |
| yl]butyl}-1,5,6,7-tetrahydroindol-4-one | | | |
| 1-{3-[4-(3,4-Dichlorophenyl)piperazin-1- | K | 10 mg/kg | subcutaneous |
| yl]propyl}-1,5,6,7-tetrahydroindol-4-one | | | |

### Example 4

### Comparative Testing

Table 4 below displays the minimum effective dose (in mg/kg) found for Compounds A-G (described above) in 3 preclinical models (performed as described above). The minimum effective dose for Compounds A-G which reversed the PCP-induced disruption of prepulse inhibition was in all cases less than the dose at which effects were seen in the conditioned avoidane response model and the locomotor activity model, both of which model unwanted side effects.

TABLE 4
Preclinical Testing of Compounds A-G

| | Prepulse | Conditioned | |
|---|---|---|---|
| | Inhibition | Avoidance | Locomotor |
| Compound | (PPI) | Response | Activity |
| A | 3 | >10 | >10 |
| B | 1 | >3 | 3 |
| C | 3 | >10 | >10 |
| D | 0.3 | 3 | 3 |
| E | 3 | >10 | >10 |
| F | 3 | >10 | >3 |
| G | 3 | >10 | >10 |

### Example 5

### Contextual Fear Conditioning in an Open Field Model

Response to the development of contextual fear conditioning in an open field model, a model for PTSD, was evaluated in C57/BL6 male mice using the Kinder Scientific MotorMonitor System (Version 3.11, Kinder Scientific, Poway, Calif.). On Day 1 (Trauma Induction Day), animals were pretreated with compound 30 minutes prior to trauma induction and placed in a cage with a shock-grid floor. Locomotor activity was evaluated by an automated open field system with infrared photo-beams. The mice were placed in the center of the cage and the following variables of motor activity were recorded: locomotor activity, fine movement, and rearing. Animals were placed in the chamber for a total of 300 seconds. After 230 seconds, a 10 second, 2.0 mA electric footshock was administered. Mice remained in the chamber for an additional minute. Mice were then injected subcutaneously for 10 consecutive days (starting 24 hours post trauma induction) with either Compound A (3.0 mg/kg) or vehicle. On day 16, following a 5 day wash out period, mice were exposed to the traumatic environment without shock for 5 minutes. Total ambulatory activity was compared between Day 1 and Day 16. Animals showing a contextual fear response on the testing day displayed less locomotor activity versus Day 1 non-shocked animals.

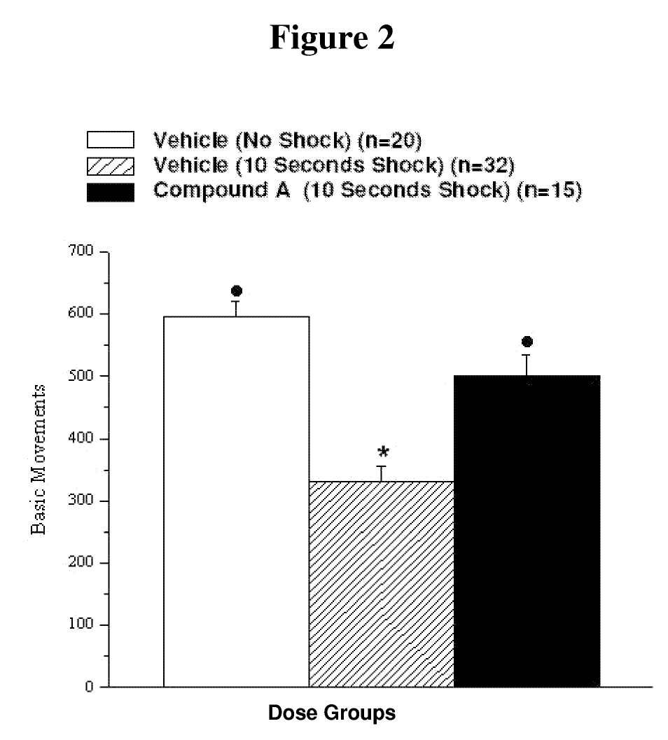

The results of the foregoing test using Compound A are illustrated in FIG. 2. Animals treated with Compound A displayed a reduction in suppressed basic movements compared to vehicle treated animals in the same environment where they received a footshock 16 days previously.

### Example 6

### Contextual Fear Conditioning in a Passive Avoidance Model

Response to the development of contextual fear conditioning in an open field model was evaluated in C57/BL6 male mice using an avoidance shuttle chamber (Kinder Scientific, Poway, Calif.). On Day 1, mice were allowed to run through the shuttle box for 3 minutes in order to acclimate them to it. One side was light and the other was dark. One Day 2, animals were dosed subcutaneously 20 minutes prior to training. Animals received a one minute acclimation in the dark, followed by a three minute training in a shuttle box with one side light and the other dark. Animals received a 1.0 mA shock when they cross to the dark side. On Days 3-5, the animals were further tested as follows. After a 1 minute acclimation period in the dark, the lights came on on one side while the other side remained dark. The latency to cross to the dark side was recorded, with a 3 minute maximum testing duration.

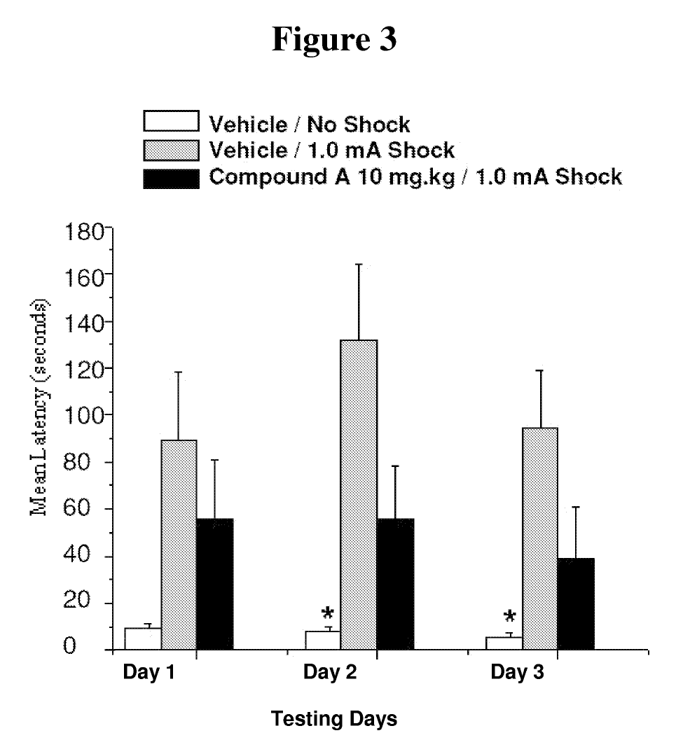

The results of the foregoing test using Compound A are illustrated in FIG. 3. Mice treated with Compound A compared to vehicle 24 hours previously showed a reduction in latency to cross over to the dark side of the chamber where they were previously shocked. This improvement in latency by Compound A was seen for three consecutive days of testing post-shock administration.

Although the present invention has been discussed in considerable detail with reference to certain preferred embodiments, other embodiments are possible. Therefore, the scope of the appended claims should not be limited to the description of preferred embodiments contained in this disclosure. All references cited herein are incorporated by reference to their entirety.

In addition, all groups described herein can be optionally substituted unless such substitution is excluded. Groupings of alternative elements or embodiments of the invention disclosed herein are not to be construed as limitations. Each group member can be referred to and claimed individually or in any combination with other members of the group or other elements found herein. It is anticipated that one or more members of a group can be included in, or deleted from, a group.

What is claimed is:

1. A method of treating post-traumatic stress disorder and acute stress disorder, comprising the step of administering to a patient in need thereof a therapeutic dose of a pharmaceutical composition comprising a compound having the following formula: 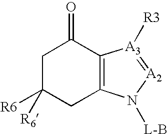
where:
(a) A₂ and A₃ are C or N;
(b) R₃ is hydrogen, alkyl, aralky, heteroaralkyl, alkenyl, aralkenyl, heteroaralkenyl, aryl, heteroaryl, or does not exist when A₃ is N;
(c) R₆ is hydrogen, alkyl, aralkyl, heteroaralkyl, aryl or heteroaryl;
(d) R₆ is hydrogen unless R₆ is alkyl, in which case R₆ is hydrogen or the same alkyl as R₆;
(e) L is a linker group; and
(f) B has the following formula: 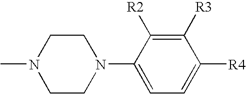
where:
(i) R2 is hydrogen, alkyl, hydroxy, halo, alkoxy, cyano, methylthio;
(ii) R3 is hydrogen, alkyl, hydroxy, methoxy, halo, alkoxy, perfluoroalkyl, nitro, amino, aminocarbonyl, aminosulfonyl;
(iii) R2 and R3 can be taken together to form a 5 or 6 member aromatic or non-aromatic ring, which can contain from 0 to 3 heteroatoms selected from the group of N, O, or S;
(iv) R₄ is hydrogen, alkyl, halo, alkoxy, perfluoroalkyl, perfluoroalkoky, or nitro; and
(v) R₃ and R₄ when taken together can form a 5 or 6 membered ring and can contain one or more heteroatoms;
and pharmaceutically acceptable salts and esters thereof.

2. The method of claim 1, wherein R₆ and R_{6′} are both hydrogen.

3. The method of claim 1, wherein R₂ is selected from the group consisting of hydrogen, halo, and alkoxy.

4. The method of claim 1, wherein R₃ is selected from the group consisting of hydrogen, alkyl, halo, alkoxy, and perfluoroalkyl.

5. The method of claim 4, wherein R₃ is trifluoromethyl.

6. The method of claim 4, wherein R₃ is halo.

7. The method of claim 1, wherein R₄ is selected from the group consisting of alkyl, halo, alkoxy, and perfluoroalkyl.

8. The method of claim 1, wherein R₂ and R₃ when taken together form a naphthalene ring.

9. The method of claim 1, wherein R₃ an R₄ are halo.

10. The method of claim 1, wherein R₂ an R₄ are halo.

11. The method of claim 1, wherein L is straight chain alkyl group of the formula —(CH₂)ₘ—, and wherein m is an integer between 1 and 6.

12. The method of claim 1, wherein the compound has the following formula:

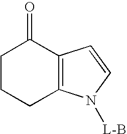

13. The method of claim 1, wherein the composition of claim 1 comprises a compound selected from the group consisting of:
1-{2-[4-(3-Chlorophenyl)piperazin-1-yl]ethyl}-1,5,6,7-tetrahydroindol-4-one;
1-{4-[4-(4-Fluorophenyl)piperazin-1-yl]butyl}-1,5,6,7-tetrahydroindol-4-one;
1-{4-[4-(4-Bromophenyl)piperazin-1-yl]butyl}-1,5,6,7-tetrahydroindol-4-one;
1-{4-[4-(3-Trifluoromethylphenyl)piperazin-1-yl]butyl}-1,5,6,7-tetrahydroindol-4-one;
1-{2-[4-(3-Trifluoromethylphenyl)piperazin-1-yl]ethyl}-1,5,6,7-tetrahydroindol-4-one;
1-{3-[4-(3-Chlorophenyl)piperazin-1-yl]propyl}-1,5,6,7-tetrahydroindol-4-one;
1-{3-[4-(3-Trifluoromethylphenyl)piperazin-1-yl]propyl}-1,5,6,7-tetrahydroindol-4-one;
1-{4-[4-(3,4-dichlorophenyl)piperazin-1-yl]butyl}-1,5,6,7-tetrahydroindol-4-one;
1-{4-[4-(3-Chloro-4-fluorophenyl)piperazin-1-yl]butyl}-1,5,6,7-tetrahydroindol-4-one;
1-{4-[4-(2,4-Dichlorophenyl)piperazin-1-yl]butyl}-1,5,6,7-tetrahydroindol-4-one; and
1-{3-[4-(3,4-Dichlorophenyl)piperazin-1-yl]propyl}-1,5,6,7-tetrahydroindol-4-one.

14. The method of claim 1, wherein the composition comprises a pharmaceutically acceptable excipient.

15. The method of claim 1, wherein the therapeutic dose is administered by an administrative route selected from the group consisting of intravenous, oral, topical, intraperitoneal, intravesical, transdermal, nasal, rectal, vaginal, intramuscular, intradermal, subcutaneous and intrathecal routes.

16. The method of claim 1, wherein the therapeutic dose is in the range of 0.0001 mg/kg to 60 mg/kg.

17. The method of claim 1, wherein the condition being treated is post-traumatic stress disorder.

18. The method of claim 1, wherein the condition being treated is acute stress disorder.
